(19) United States
(12) Patent Application Publication
(10) Pub. No.: US 2025/0319101 A1
(43) Pub. Date: Oct. 16, 2025

(54) ORAL PHARMACEUTICAL COMPOSITIONS OF PROGESTERONE AND ESTRADIOL

(71) Applicant: CHEMO RESEARCH, S.L., Madrid (ES)

(72) Inventors: Costoya Mujico ALEJANDRO, Madrid (ES); Moya Ortega MARÍA DOLORES, Madrid (ES)

(21) Appl. No.: 18/698,961

(22) PCT Filed: Oct. 7, 2022
(86) PCT No.: PCT/EP2022/077936

§ 371 Date: Apr. 5, 2024

(30) Foreign Application Priority Data
Oct. 8, 2021 (EP) 21382903.9

(51) Int. Cl. A61K 31/57; A61K 9/48; A61K 31/565

(52) U.S. Cl. CPC A61K 31/57; A61K 9/4858; A61K 31/565

(57) ABSTRACT

Novel oral and stable pharmaceutical compositions comprising progesterone or a pharmaceutically acceptable salt thereof in combination with estradiol or a pharmaceutically acceptable salt thereof are provided. The present invention provides novel self-emulsifying systems for pharmaceutical compositions comprising progesterone or pharmaceutically acceptable salt thereof in combination with estradiol, or pharmaceutically acceptable salt thereof which provides good solubility of the two APIs, and/or reduces their bio-degradation. The invention also refers to a process for preparing said pharmaceutical compositions and to the use of said pharmaceutical compositions.

### CROSS-REFERENCE TO RELATED PATENT APPLICATIONS

This is a U.S. National Phase Application under 35 U.S.C. § 371 of International Patent Application No. PCT/EP2022/077936, filed Oct. 7, 2022, and claims priority to European Patent Application No. 21382903.9, filed Oct. 8, 2021, which is incorporated by reference in its entirety. The International Application was published on Apr. 13, 2023, as International Publication No. 2023/057626 A1.

### FIELD OF THE INVENTION

The present application relates to novel oral and stable pharmaceutical compositions comprising progesterone or a pharmaceutically acceptable salt thereof in combination with estradiol or a pharmaceutically acceptable salt thereof, to the process for preparing said pharmaceutical compositions and to the use of said pharmaceutical compositions.

### STATE OF THE ART

Hormone replacement therapy (HRT) is a medical treatment that involves the use of one or more of a group of medications designed to increase or supplement hormone levels in women who lack adequate hormone production.

HRT reduces and prevents symptoms caused by a decrease in circulating estrogen and progesterone hormones, regardless as to whether the subject is pre-menopausal, peri-menopausal, menopausal or post-menopausal. However, there may be specific symptoms during the progression of each stage of menopause.

Many postmenopausal women are treated with hormone therapy (HT) in an attempt to alleviate symptoms of menopause, which are primarily hot flashes, night sweats, calcium loss from bone, vaginal atrophy, and also to prevent osteoporosis and for the prevention of heart diseases. Estrogen is the principal hormone used to treat postmenopausal symptoms. A variety of estrogenic preparations are available, including the natural endogenous estrogen, 17b-estradiol. However, the prolonged use of unopposed estrogens increases the risk of endometrial hyperplasia and endometrial cancer.

To prevent certain undesirable side effects of estrogen administration, such as endometrial hyperplasia and to reduce the incidence of endometrial cancer, a progestogen is used either continuously combined or sequentially with the estrogens. The progestogens available for therapeutic use are synthetic progestogens (progestins), and also natural progestogen (progesterone).

It is desirable to use estradiol and progesterone to treat a variety of endocrine disorders, and also for use as a contraceptive. However, it is well known that neither of these compounds are suitable for oral administration due to the manner in which they are absorbed from the digestive system. In particular, these steroidal hormones are carried by the portal system to the liver, where they are rapidly metabolized into inactive metabolites. Consequently, effective oral administration has required excessively high dosage levels to compensate for the metabolic breakdown of these compounds.

While combining both estradiol and progesterone in a single dosage form may be considered ideal for therapeutic reasons and convenient for patients, the difference in chemical structure between the compounds and their poor aqueous solubility present challenges in producing formulations with the appropriate bioavailability.

USFDA first approved a drug product in the form of oral soft-gelatine capsule containing bio-identical estradiol and bio-identical progesterone in October 2018, which is commercially available as BIJUVA® (TherapeuticsMD, Boca Raton, FL), and which is indicated for the treatment of moderate to severe vasomotor symptoms due to menopause in post-menopausal women. Each capsule of BIJUVA® contains estradiol, progesterone, medium chain mono and di-glycerides, medium chain triglycerides, ammonium hydroxide, ethanol, ethyl acetate, FD&C Red #40, gelatine, glycerine, hydrolyzed gelatine, isopropyl alcohol, lecithin, lauroyl polyoxyl-32 glyceride, polyethylene glycol, polyvinyl acetate phthalate, propylene glycol, purified water and titanium dioxide.

U.S. Pat. No. 8,633,178 of TherapeuticsMD describes pharmaceutical compositions comprising progesterone, estradiol and solubilizing agent, wherein the solubilizing agent comprises medium chain oil (C6-C12 oil). Specifically pharmaceutical compositions in capsule form comprising progesterone and estradiol and CAPMUL MCM (medium chain mono and diglycerides) as oil and GELUCIRE 44/14 (Lauroyl macrogol 32-glycerides) as surfactant are disclosed as providing the desire solubility of both APIs. The process for manufacture the capsules comprising progesterone, estradiol, medium chain oils and a surfactant involves mixing CAPMUL MCM oil (Medium Chain Mono- and Diglycerides) and GELUCIRE 44/14 surfactant under heating to 40° C. (Example 12), or under heating to 65° C. (Example 14, CAPMUL MCM oil and GELUCIRE 44/14 surfactant), and then mixing estradiol, and finally heat may be removed and progesterone is added (see scheme in FIG. 1). US 2017/281646 discloses capsules comprising progesterone, estradiol, Kolliphor RH40, Capmul MCM, polysorbate 80 and Capmul 708G. The capsules are used for treating vasomotor symptoms of menopause and they are prepared by mild heating, that is from about 35° C. to 60° C., preferably 40° C. The compositions are prepared by combining the ingredients using standard preparatory techniques.

Patent application WO 2021/081276 of Slayback discloses a stable pharmaceutical composition suitable for oral administration and comprising progesterone, optionally estradiol, a solubilizing agent that is a long-chain oil and a surfactant, optionally an antioxidant and optionally a co-solvent, preferably wherein the solubilizing agent and the surfactant are present in the composition in a weight ratio of 50:50 to 99:1. Slayback process for preparing progesterone and estradiol capsules comprises heating the long-chain oil and the surfactant until dissolved completely, adding the co-solvent, optionally the anti-oxidant, and then adding progesterone and finally estradiol to obtain a final suspension. In all the examples, Slayback process involves mixing long-chain oils and surfactant under heating at 40° C.

As generally known, drawbacks for the progesterone formulations disclosed in the prior art are presence of Impurity M, a degradant impurity of progesterone that appears under storage.

It was reported in a non-clinical review document submitted to the FDA by TherapeuticsMD that Impurity-M was observed above the ICH identification limit of 0.2% on accelerated stability conditions of Phase 3 clinical batches (see page 16 of Non-Clinical Review document of BIJUVA®).

Secondly, Slayback discloses that lower amount of impurity M was detected. However, still 0.016% impurity M when stored 40° C., 75% HR, for 3 months is detected (Composition 30), and 0.020% impurity M when stored 40° C., 75% HR, for 3 months is detected (Composition 31) (see related substance data of progesterone, Example 16).

One of the main drawbacks for the progesterone and estradiol capsule formulations disclosed in the prior art is that they need to be prepared by heating the oil and/or the oil/surfactant mixture at 40° C. to 65° C. In Bijuva, medium chain oils are solid at room temperature and thus, needs to be heated. Long-chain oils are liquid at room temperature and despite that, when co-formulated with surfactants, viscosity is high, and heating is also required about 40° C.

WO 97/40823 discloses capsules comprising progesterone, estradiol, coconut oil, Cremophor RH40, Inwitor 988 and ethanol. The capsules are used for treating menopausal symptoms but no specific reference is made to vasomotor instability. The capsules are prepared by known methods in the art.

US 2003/104048 and WO 03/068186, both disclose capsules comprising progesterone, estradiol, Cremophor EL, Labrafil M2125CS, vitamin E TPGS, and propylene glycol. WO 03/068186 discloses that the capsules are prepared by encapsulating estradiol and a part of progesterone in a small two piece hard gelatin capsule, that is next encapsulated in a larger two-piece hard gelatin capsule comprising the remaining part of progesterone solubilized in Cremophor EL, Labrafil M2125CS, vitamin E TPGS, and propylene glycol.

Developing stable oral compositions of 17b-estradiol and progesterone in a single dosage form has always been challenging because of their differences in structure and solubility.

Thus, an object of the present invention is the provision of a stable pharmaceutical composition for oral administration comprising 17b-estradiol and progesterone having good solubility and appropriate and/or improved bioavailability.

Furthermore, and additionally, it would be desirable that the compositions comprising progesterone in combination with estradiol do not reveal the presence of M-impurity under storage.

Thus, it is a further object of the present invention that said novel stable pharmaceutical composition comprising 17b-estradiol and progesterone do not present Impurity M under storage conditions.

Further, it was found that the manufacturing conditions necessary to produce such stable pharmaceutical composition comprising 17b-estradiol and progesterone are highly advantageous in terms of energy consumption.

### SUMMARY OF THE INVENTION

These objectives are achieved with the present invention that, in a first aspect thereof, relates to a stable pharmaceutical composition comprising a capsule dosage form suitable for oral administration, the composition comprising:
progesterone or a pharmaceutically acceptable salt thereof;
estradiol or a pharmaceutically acceptable salt thereof;
a long-chain oil selected from the group consisting of Grapeseed oil, Mixture of refined soybean oil, glyceryl distearate and polyglyceryl-3 dioleate, Wheat germ oil, Cottonseed oil, Rice bran oil, Glyceryl monolinoleate, Soybean oil, Glyceryl monooleate, Olive oil;
a surfactant selected from the group consisting of Linoleoyl Polyoxyl-6 glycerides, Capryolcaproyl polyoxyl-8-glycerides, Polysorbate 80, polyoxyl-40 hydrogenated castor oil, Oleoyl polyoxyl-6 glycerides, Polyoxyl-35 castor oil, Polyoxyl-15 Hydrostearate;
a co-surfactant selected from the group consisting of propylene glycol monocaprylate, Propylene glycol monolaurate, polyglyceryl-3 dioleate; and
a co-solvent.

In a second aspect thereof, the invention relates to a stable pharmaceutical composition as defined in the first aspect, wherein long-chain oil, surfactant and co-surfactant are present together as a self-emulsifying system. The present invention provides novel self-emulsifying systems for pharmaceutical compositions comprising progesterone or pharmaceutically acceptable salt thereof in combination with estradiol, or pharmaceutically acceptable salt thereof capable of improving the solubility of the two APIs and decreasing their bio-degradation.

In a third aspect thereof, the invention relates to a process for preparing said stable pharmaceutical composition as defined in the first and second aspects. The present invention provides a novel process for preparing said stable pharmaceutical compositions which is capable of using mild and environmentally friendly conditions. The authors of the present invention have found that preparing a preconcentrate of estradiol is a new strategy allowing the process to be performed at room temperature or at least at lower temperature conditions compared with the prior art. Advantageously, the process disclosed herein is more cost-effective, environmentally friendly and energetically sustainable.

According to a further aspect thereof, this invention relates to a stable pharmaceutical composition suitable for oral administration obtainable by a process as defined in the third aspect of the invention. Advantageously, the obtainable composition exhibits no presence of Impurity-M when stored for at least 3 months at 40° C./75% relative humidity (RH) condition.

In still a further aspect thereof, the invention relates to said stable pharmaceutical compositions for use in the treatment of moderate to severe vasomotor symptoms (VMS) related to menopause, for use in the treatment of secondary amenorrhea and/or for use in the prevention of endometrial hyperplasia in non-hysterectomied postmenopausal women who are receiving conjugated estrogen.

Another aspect relates to methods of treatment using said stable pharmaceutical compositions, the method comprising orally administering an effective amount of said pharmaceutical compositions at a desired dosage regime.

Following the administration of the stable pharmaceutical composition of the invention comprising progesterone and estradiol as defined in claim 1 to a subject, the concentration and metabolism of progesterone or estradiol can be measured in a sample (e.g., a blood, serum, or plasma sample) from the subject. Progesterone is metabolized to pregnanediols and pregnanolones, which are then conjugated to glucuronide and sulfate metabolites that are excreted or further recycled. Estradiol is converted reversibly to estrone, and both estradiol and estrone can be converted to the metabolite estriol.

Specifically provided is a method for treating vasomotor symptoms (VMS) related to menopause in a human patient, the method comprising orally administering an effective amount of said pharmaceutical compositions at a desired dosage regime. Also described is a method for preventing endometrial hyperplasia in non-hysterectomized postmenopausal women who are receiving conjugated estrogen. Further described is a method for treating secondary amenorrhea in a human patient.

### BRIEF DESCRIPTION OF THE DRAWINGS

FIG. 1 illustrates the dissolution profile (% release) of Estradiol release from RLD BIJUVA in dissolution media (0.1 N HCl) compared with formulations ESPR-0437-42, ESPR-0437-46-1 and ESPR-0437-46-2 (from Example 3.1).

FIG. 2 illustrates the dissolution profile (% release) of Progesterone release from RLD BIJUVA in dissolution media (0.1 N HCl) compared with formulations ESPR-0437-42, ESPR-0437-46-1 and ESPR-0437-46-2 (from Example 3.1).

FIG. 3 illustrates the dissolution profile (% release) of Estradiol release from RLD BIJUVA in dissolution media (0.1 N HCl) compared with formulation ESPR-0437-42 (from Example 3.1), and also compared with formulations ESPR-0437-50-1, ESPR-0437-50-2, ESPR-0437-53-1 and ESPR-0437-53-2 (from Example 3.1).

FIG. 4 illustrates the dissolution profile (% release) of Progesterone release from RLD BIJUVA in dissolution media (0.1 N HCl) compared with formulation ESPR-0437-42 (from Example 3.1), and also compared with formulations ESPR-0437-50-1, ESPR-0437-50-2, ESPR-0437-53-1 and ESPR-0437-53-2 (from Example 3.1).

FIG. 5 illustrates the dissolution profile (% release) of Estradiol release from RLD BIJUVA in dissolution media (0.1 N HCl) compared with formulations ESPR-0437-27-1, ESPR-0437-47-1, ESPR-0437-47-2 and ESPR-0437-49 (from Example 3.2).

FIG. 6 illustrates the dissolution profile (% release) of Progesterone release from RLD BIJUVA in dissolution media (0.1 N HCl) compared with formulations ESPR-0437-27-1, ESPR-0437-47-1, ESPR-0437-47-2 and ESPR-0437-49 (from Example 3.2).

FIG. 7 illustrates the chromatogram of sample solution of Labrasol formulation (from Example 1), wherein no presence of Impurity-M under storage is detected.

FIG. 8 illustrates the chromatogram of spiked sample solution with Progesterone impurities signals present in the sample.

FIG. 9 illustrates the chromatogram of sample solution of Kolliphor formulation (from Example 2), wherein no presence of Impurity-M under storage is detected.

FIG. 10 illustrates the chromatogram of spiked sample solution with Progesterone impurities signals present in the sample.

FIG. 11 illustrates the chromatogram of Progesterone impurities in which Impurity M signals at 22.723.

FIG. 12: illustrates the Mean Baseline-adjusted Plasma Progesterone Concentration-Time Profiles (Linear Scale, A: n=28/B: n=29/C: n=26), A refers to Estradiol/Progesterone 1 mg/100 mg capsules, according to Example 1 of the application; B refers to Estradiol/Progesterone 1 mg/100 mg capsules, according to Example 2 of the application; and C refers to Bijuva® (Estradiol and Progesterone) 1 mg/100 mg capsules, Batch No.: 3551122A.

FIG. 13: illustrates the Mean Baseline-adjusted Plasma Total Estrone Concentration-Time Profiles (Linear Scale, A: n=28/B: n=30/C: n=28), A refers to Estradiol/Progesterone 1 mg/100 mg capsules, according to Example 1 of the application; B refers to Estradiol/Progesterone 1 mg/100 mg capsules, according to Example 2 of the application; and C refers to Bijuva® (Estradiol and Progesterone) 1 mg/100 mg capsules, Batch No.: 3551122A.

### DETAILED DESCRIPTION OF THE INVENTION

All terms used in this application, unless otherwise specified, are to be understood in their ordinary meaning as known in the technical field. Other more specific definitions of certain terms used in this application are listed below and are intended to be applied uniformly to the entire application, unless indicated otherwise.

It is to be understood that this invention is not limited to particularly exemplified materials or process parameters as such may, of course, vary. It is also to be understood that the terminology used herein is for the purpose of describing particular embodiments of the invention only, and is not intended to be limiting.

The singular forms “a,” “an,” and “the” include plural referents unless the context clearly dictates otherwise.

As used herein the terms “composition” and “formulation” refer to a pharmaceutical composition administered to a patient in need of treatment in the form of a capsule dosage form.

The term “stable” refers to both the physical and chemical stability of a composition in any form, such as a suspension. A composition is said to be stable if it exhibits minimal change over time relative to when it is manufactured. Stability is measured at various time points through a planned product expiration date with evaluation criteria including such items as appearance, phase separation between solubilizing agent and surfactant, pH of composition, content of active ingredient(s), and levels of degradation products, impurities, or related substances.

As used herein, the terms “good stability” refers to the avoidance of estradiol and progesterone precipitation during storage and under stress conditions.

As used herein the term “progesterone” refers to progesterone free base or a pharmaceutically acceptable salt, solvate, anhydrous, hemihydrate, hydrate, co-crystal or polymorph thereof. As used herein, progesterone refers to the bio-identical or body-identical form of progesterone found in the human body.

The term “micronized progesterone”, as used herein, includes micronized progesterone having a D99 particle size value below about 60 microns, further below about 40 microns and having D90 particle size value below about 30 microns, further below about 20 microns and having D65 particle size value below about 25 microns, further below about 10 microns.

The term “estrogen” refers to a group of several female sex hormones produced primarily by the ovaries, including estradiol, estrene, and estriol. As used herein, unless otherwise specified, estrogen refers to estradiol.

The term “estradiol” refers to (17beta)-estra-1,3,5(10)-triene-3,17-diol. Estradiol is also interchangeably called 17beta-estradiol, oestradiol, or E2, and is found endogenously in the human body. As used herein, estradiol refers to the bio-identical or body-identical form of estradiol found in the human body. Estradiol is supplied in an anhydrous or hemi-hydrate form. Estradiol may be micronized or not. For the purposes of this disclosure, the anhydrous form or the hemihydrate form can be substituted for the other by accounting for the water or lack of water according to well-known and understood techniques.

The term “long-chain” is used to describe the aliphatic chain length of fatty acid containing molecules. The term “long-chain” as used herein means any long-chain carbon-containing substance, including C₁₆-C₂₄ fatty acid esters of glycerol, fatty acids, and mono-, di-, and tri-glycerides of such substances. Particular “long-chain” oils are desirable for the subject of the pharmaceutical composition disclosed herein. Said oils would suspend and/or solubilize any suitable active ingredients described herein.

The term “bioavailability” refers to the rate and extent to which an active ingredient or active moiety is absorbed from a drug product and becomes available at the site of action. For example, bioavailability can be measured as the amount of active ingredient in the blood (serum or plasma) as a function of time. Pharmacokinetic (PK) parameters such as AUC, Cmax, or Tmax may be used to measure and assess bioavailability.

The term “bioequivalent” refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. In practice, two products are considered bioequivalent if the 90% confidence interval of the AUC or Cmax is within 80.00% to 125.00%.

The terms “bio-identical hormone” and “body-identical” refer to an active pharmaceutical ingredient that is structurally identical to a hormone naturally or endogenously found in the human body (e.g., estradiol or progesterone).

As used herein, the term “prevent” refers to the prophylactic treatment of a subject who is at risk of developing a condition (e.g., steroid hormone deficiency) resulting in a decrease in the probability that the subject will develop the condition.

As used herein, the phrase “steroid hormone” refers to progesterone, 17-hydroxyprogesterone, 5α-dihydroprogesterone, and estradiol.

As used herein, the terms “room temperature” refers to the ambient temperature, a temperature typically comprised between 15° C. and 30° C., preferable between 20° C. and 25° C.

In its first aspect, the invention relates to a novel stable pharmaceutical composition comprising a capsule dosage form suitable for oral administration, the composition comprising:
progesterone or a pharmaceutically acceptable salt thereof;
estradiol or a pharmaceutically acceptable salt thereof;
a long-chain oil selected from the group consisting of Grapeseed oil, Mixture of refined soybean oil, glyceryl distearate and polyglyceryl-3 dioleate, Wheat germ oil, Cottonseed oil, Rice bran oil, Glyceryl monolinoleate, Soybean oil, Glyceryl monooleate, Olive oil;
a surfactant selected from the group consisting of Linoleoyl Polyoxyl-6 glycerides, Capryolcaproyl polyoxyl-8-glycerides, Polysorbate 80, polyoxyl-40 hydrogenated castor oil, Oleoyl polyoxyl-6 glycerides, Polyoxyl-35 castor oil, Polyoxyl-15 Hydrostearate;
a co-surfactant selected from the group consisting of propylene glycol monocaprylate, Propylene glycol monolaurate, polyglyceryl-3 dioleate; and
a co-solvent.

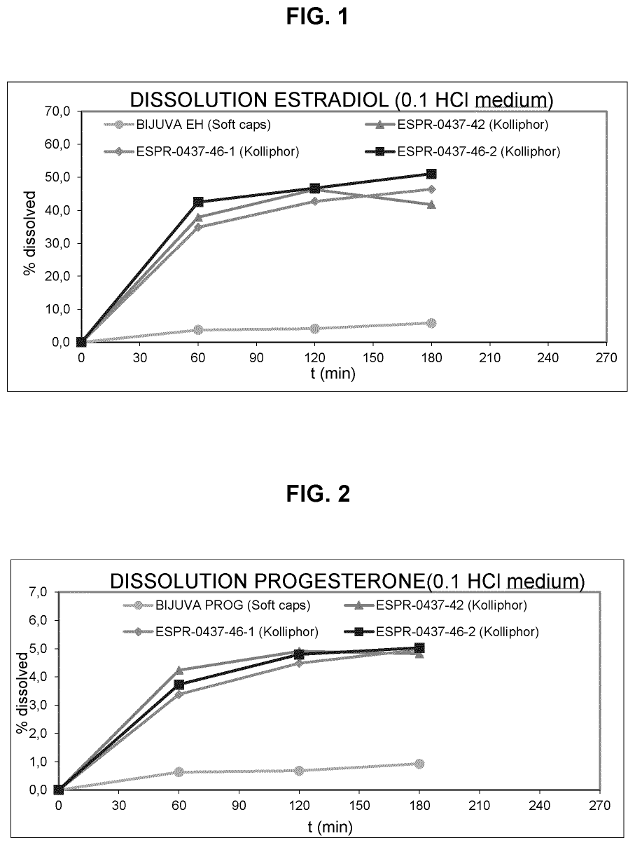

Said composition shows improved solubility of both APIs in emulsion media. Furthermore, said composition also shows good stability. Surprisingly, the co-surfactant in the composition adds a new role in this type of compositions. It has been found that when the co-surfactant is present in combination with the long-chain oil and the surfactant, solubility of both of the APIs remains similar in respect to the formulations without the presence of a co-surfactant, but this one positively influences in the dissolution profile. Different formulations containing or not containing co-surfactant were tested for solubility and dissolution in 0.1 N HCl medium (Example 3 and FIGS. 1 to 6). It was found that the co-surfactant allows modulating the dissolution profile, and also forming self-emulsifying systems together with the long-chain oil and the surfactant.

It is known that poor aqueous solubility, oral bioavailability, inter, and inter-subject variability, and physical stability have always been a concern for pharmaceutical formulation scientists while formulating an oral dosage form. Self-Emulsifying Drug Delivery System (SEDDS) is a promising new approach to mitigating those potential problems. The main advantages of SEDDS are that it increases the solubility and decreases the bio-degradation of lipophilic drugs. Mostly BCS II & IV Class drugs, as progesterone and estradiol, are preferable. SEDDS is an admixture of drugs, oil, surfactants, co-solvents, and stabilizers. With little energy input, they form (o/w) microemulsion within the gastrointestinal lumen.

In its second aspect, the invention relates to a stable pharmaceutical composition as defined in the first aspect, wherein long-chain oil, surfactant and co-surfactant are present together as a self-emulsifying system. The self-emulsifying system comprises a hydrophilic surfactant, a lipophilic co-surfactant and a long-chain oil as vehicle.

Preferably, the stable pharmaceutical composition according to the first and the second aspects of the invention comprises:
30-39.5% w/w of composition as active ingredients including progesterone or a pharmaceutically acceptable salt thereof, and estradiol or a pharmaceutically acceptable salt thereof;
60-69.5% w/w of composition as a self-emulsifying system including long-chain oil, surfactant and co-surfactant; and
0.5-5% w/w of composition as a co-solvent,
   wherein the sum of the percentages does not exceed 100%.

The preferable stable pharmaceutical composition comprises:
30-38% w/w of composition as active ingredients including progesterone or a pharmaceutically acceptable salt thereof, and estradiol or a pharmaceutically acceptable salt thereof;
60-68% w/w of composition as a self-emulsifying system including long-chain oil, surfactant and co-surfactant; and
0.5-5% w/w of composition as a co-solvent,
   wherein the sum of the percentages does not exceed 100%.

In an embodiment, the active ingredients are in an amount between 32-38% w/w of the composition.

In an embodiment, the self-emulsifying system including long-chain oil, surfactant and co-surfactant is in an amount between 62-68% w/w of the composition.

In an embodiment, the co-solvent is in an amount between 0.5-3% w/w of the composition.

Preferably, the co-solvent is in an amount between about 0.8 to about 2.5% w/w of the composition, and more preferably about 1% w/w of the composition.

Preferably, progesterone or a pharmaceutically acceptable salt thereof is in an amount between 30 and 39.9% w/w of the composition, more preferable between 32 and 35% w/w of the composition, and estradiol or a pharmaceutically acceptable salt thereof is in an amount between 0.1 and 10% w/w of the composition, more preferable between 0.12 and 5% w/w of the composition. Preferable progesterone is progesterone micronized. Preferable estradiol is estradiol hemihydrate. Preferable composition comprises progesterone micronized in combination with estradiol hemihydrate

The long-chain oils include the group consisting of Grapeseed oil, Mixture of refined soybean oil, glyceryl distearate and polyglyceryl-3 dioleate (Geloil SC), Wheat germ oil, Cottonseed oil, Rice bran oil, Glyceryl monolinoleate (Maisine CC), Soybean oil, Glyceryl monooleate, Type 40 (Peceol), Olive oil. Still more preferable long-chain fatty acid ester of glycerol is Glyceryl monolinoleate (Maisine CC). The names used herein are in accordance the nomenclature of US Pharmacopeia.

The surfactant is selected from the group consisting of Linoleoyl Polyoxyl-6 glycerides (Labrafil 2125), Capryolcaproyl polyoxyl-8-glycerides (Labrasol), Polysorbate 80 (Tween 80), polyoxyl-40 hydrogenated castor oil (Kolliphor RH 40), Oleoyl polyoxyl-6 glycerides (Labrafil 1944), Polyoxyl-35 castor oil (Kolliphor EL), Polyoxyl 15 Hydrostearate (Kolliphor HS-15). The names used herein are in accordance the nomenclature of US Pharmacopeia. Preferable surfactants are Capryolcaproyl polyoxyl-8-glycerides (Labrasol) or polyoxyl-40 hydrogenated castor oil (Kolliphor RH 40).

The co-surfactant is selected from the group consisting of propylene glycol monocaprylate, type II (Capryol 90), Propylene glycol monolaurate, type II (Lauroglycol 90), polyglyceryl-3 dioleate (Plurol Oleique). The names used herein are in accordance the nomenclature of US Pharmacopeia. Preferable co-surfactants are propylene glycol monocaprylate, type II (Capryol 90) or polyglyceryl-3 dioleate (Plurol Oleique).

In an embodiment, the co-solvent is selected from the group consisting of PEG-400, Ethanol, Transcutol HP. Preferably, the co-solvent is Ethanol (Absolute) or Transcutol HP. Still more preferable, the co-solvent is Ethanol (Absolute).

In one preferred embodiment, the stable pharmaceutical composition comprising a capsule dosage form includes estradiol hemihydrate, progesterone micronized, glyceryl monolinoleate (MAISINE CC), Capryolcaproyl polyoxyl-8-glycerides (LABRASOL), propylene glycol monocaprylate. (CAPRYOL 90) and ethanol (Absolute). Preferably, in this embodiment, estradiol is estradiol hemihydrate. Also preferably, in this embodiment, progesterone is progesterone micronized.

In this embodiment, a preferable capsule dosage form includes the following unitary composition:

Unitary composition (1) (E₁)

| | Unitary | (%) w/w of the |
|---|---|---|
| Material | formula (mg) | composition |
| Progesterone Micronized | 100.0 | 33.34 |
| Estradiol Hemihydrate | 1.0 | 0.34 |
| Maisine CC | 17.6 | 5.88 |
| Labrasol | 115.6 | 38.53 |
| Capryol 90 | 62.7 | 20.90 |
| Ethanol Absolute | 3.0 | 1.00 |
| Total | 300.0 | 100.0 |

Another preferable capsule dosage form includes the following unitary composition:

Unitary composition (2) (EG)

| | Unitary | (%) w/w of the |
|---|---|---|
| Material | formula (mg) | composition |
| Progesterone Micronized | 100.0 | 33.33 |
| Estradiol Hemihydrate | 1.0 | 0.34 |
| Maisine CC | 137.2 | 45.73 |
| Labrasol | 29.4 | 9.80 |
| Capryol 90 | 29.4 | 9.80 |
| Ethanol Absolute | 3.0 | 1.00 |
| Total | 300.0 | 100.0 |

In another embodiment, the stable pharmaceutical composition comprising a capsule dosage form includes estradiol hemihydrate, progesterone micronized, glyceryl monolinoleate (MAISINE CC), polyoxyl-40 hydrogenated castor oil (KOLLIPHOR RH 40), polyglyceryl-3 dioleate (PLUROL OLEIQUE) and ethanol (Absolute). Preferably, in this embodiment, estradiol is estradiol hemihydrate. Also preferably, in this embodiment, progesterone is progesterone micronized.

In this embodiment, preferable capsule dosage form includes the following unitary composition:

Unitary composition (3) (E₂)

| | Unitary | (%) w/w of the |
|---|---|---|
| Material | formula (mg) | composition |
| Progesterone Micronized | 100.0 | 33.33 |
| Estradiol Hemihydrate | 1.0 | 0.34 |
| Maisine CC | 98.0 | 32.67 |
| Kolliphor RH40 | 19.5 | 6.50 |
| Plurol Oleique CC | 78.5 | 26.16 |
| Ethanol Absolute | 3.0 | 1.00 |
| Total | 300.0 | 100.0 |

The oral and stable pharmaceutical composition of the present invention can be encapsulated in a gelatine capsule, or other similar encapsulated dosage form known to those of skill in the art. The gelatine capsule can be a soft gelatine capsule or a hard gelatine capsule. The hard gelatine capsule can be a two-piece capsule suitable for liquid filling, which typically includes a first capsule portion (i.e., half or bottom) and a second capsule portion (i.e., the other half or top). The soft gelatine capsule can be a two-piece capsule wherein two portions are sealed together or a one-piece, hermetically sealed capsule. Preferable capsule dosage form is a soft gelatine capsule.

The capsule dosage form of the invention may, if desired, include one or more other excipients, such as but not limited to colorants, flavoring agents, and preservatives. The choice of excipients will, to a large extent, depend on factors such as the particular mode of administration, the effect of the excipients on solubility and stability, and the nature of the dosage form.

The capsule dosage form of the invention comprises the combination of progesterone and estradiol in a therapeutically effective dosage amount of 100 mg of progesterone micronized and 1 mg of estradiol hemihydrate. In an alternatively embodiment, the capsule dosage form of the invention comprises a therapeutically effective dosage amount of 100 mg of progesterone micronized and 0.5 mg of estradiol hemihydrate. The capsule dosage form of the invention has a total weight of 300 mg.

In this second aspect, the invention relates to a self-emulsifying system. Specifically, three self-emulsifying systems have been identified to be used with progesterone or a pharmaceutically acceptable salt thereof in combination with estradiol or a pharmaceutically acceptable salt thereof.

The authors of the present invention have found that when a certain ratio of long-chain oil:surfactant:co-surfactant is present in the composition, an stabilized Self-Emulsifying Drug Delivery System (SEDDS) is provided while maintaining the good solubility of the APIs in the formulation. Furthermore, said ratios determines the percentages of estradiol and of progesterone dissolved in the medium (See Example 3 and FIGS. 1 to 6).

Preferable self-emulsifying system consists of a long chain oil, a surfactant and a co-surfactant.

Advantageously, the formulation of an self-emulsifying system consisting of long chain oil, surfactant and co-surfactant in accordance with the present invention is capable of improving the absorption of the two drugs via the lymphatic system.

It is known that glycerides with unsaturated long chain fatty acid are absorbed via the lymphatic system that is because after digestion, triglycerides are transformed into monoglycerides and fatty acids, which are then absorbed into the enterocyte. Medium chain lipids, with fatty acid chain length below 12, diffuse across the enterocyte directly into the blood circulation. Long chain lipids, with fatty acid chain length equal to or above 12, are reassembled into triglycerides in the endoplasmic reticulum. Here they are combined with proteins to form chylomicrons. These vesicles are too large to access the blood capillary. As the interstitial lymph capillary allows larger molecules to penetrate, they are transferred to the lymph via a passive diffusion.

In a preferred embodiment of this aspect, the invention relates to a first self-emulsifying system E₁ that provides in use an emulsion formation that does not show phase separation in the gastrointestinal fluid. The self-emulsifying system E₁ includes a surfactant which is liquid at room temperature.

The self-emulsifying system E₁ includes:
5-45% wt of a long-chain oil;
45-85% wt of a surfactant; and
2-35% wt of a co-surfactant,
   wherein the sum of the percentages does not exceed 100%.

Surprisingly, the self-emulsifying system E₁ provides homogeneous and intense milky emulsions. For an improved emulsion formation is required at least 50% of surfactant which it can be fixed to 45% w/w of the self-emulsifying system. At lower surfactant concentration, different formation or slight emulsions appear when water is added. An amount of co-surfactant higher than 35% could lead to loss of emulsification.

Preferable self-emulsifying system E₁ comprises long-chain oil in an amount 9-40% wt.

Preferable self-emulsifying system E₁ comprises surfactant in an amount 50-60% wt.

Preferable self-emulsifying system E₁ comprises co-surfactant in an amount 10-32% wt.

Examples of self-emulsifying system E₁ can be found in Table 5 included below (see examples ESPR-0437-27-1, ESPR-0437-27-2, ESPR-0437-27-3).

A preferable capsule dosage form including the self-emulsifying system E₁ corresponds to the unitary composition (1).

In another preferred embodiment of this aspect, the invention relates to a second self-emulsifying system EG that provides in use an emulsion-gel (EG) formation that does not show phase separation in the gastrointestinal fluid. The self-emulsifying system EG includes a surfactant which is liquid at room temperature.

The self-emulsifying system EG includes:
40-80% wt of a long-chain oil;
10-50% wt of a surfactant; and
2-25% wt of a co-surfactant,
   wherein the sum of the percentages does not exceed 100%.

The self-emulsifying system EG also provides the emulsion formation. Although this self-emulsifying system EG is limited because gel formation outside the ranges is observed, solution flipping lead to emulsion formation. This self-emulsifying system EG compared to the self-emulsifying system E₁ is expanded to lower surfactant concentration and to higher long-chain oil concentration which indicate slight emulsion. The increment of co-surfactant amount above 25% could lead to loss of emulsification.

Preferable self-emulsifying system EG comprises long-chain oil in an amount 55-70% wt.

Preferable self-emulsifying system EG comprises surfactant in an amount 15-40% wt.

Preferable self-emulsifying system EG comprises co-surfactant in an amount 5-20% wt.

Examples of self-emulsifying system EG can be found in Table 6 included below (see examples ESPR-0437-47-1, ESPR-0437-47-2, ESPR-0437-49).

A preferable capsule dosage form including the self-emulsifying system EG corresponds to the unitary composition (2).

In still another preferred embodiment of this aspect, the invention relates to a third self-emulsifying system E₂ that provides in use an emulsion formation that does not show phase separation in the gastrointestinal fluid. The self-emulsifying system E₂ includes a surfactant which is solid at room temperature.

The self-emulsifying system E₂ includes:
20-60% wt of a long-chain oil;
8-40% wt of a surfactant; and
18-55% wt of a co-surfactant,
   wherein the sum of the percentages does not exceed 100%.

The self-emulsifying system E₂ also provides the emulsion formation. However, the increment of surfactant amount above 40% could lead to loss of emulsification.

Preferable self-emulsifying system E₂ comprises long-chain oil in an amount 44-55% wt.

Preferable self-emulsifying system E₂ comprises surfactant in an amount 10-15% wt.

Preferable self-emulsifying system E₂ comprises co-surfactant in an amount 35-40% wt.

Examples of self-emulsifying system E₂ can be found in Table 4 (Example 3) included below (see examples system E₂ ESPR-0437-53-1 and ESPR-0437-53-2, versus comparative examples ESPR-0437-50-1, and ESPR-0437-50-2).

A preferable capsule dosage form including the self-emulsifying system E₂ corresponds to the unitary composition (3).

Since the stable pharmaceutical composition comprising capsule dosage form suitable for oral administration of the invention includes 60-69.5%, preferable 60-68%, more preferable 62-68% by weight of self-emulsifying system, it is of general knowledge for a person skilled in the art to perform the calculations to know the amount of long-chain oil, surfactant and co-surfactant referred to in the composition for all the three self-emulsion systems described above. It is evident to a person skilled in the art that by varying the range of the self-emulsifying system, the range of the active ingredients and/or the range of the co-solvent of the pharmaceutical composition of the first aspect will also vary within the ranges indicated above, so that the sum of the percentages of two active ingredients, the self-emulsifying system and the co-solvent does not exceed 100%.

In a further aspect, the invention relates to a stable pharmaceutical composition comprising capsule dosage form suitable for oral administration that shows good stability in terms of progesterone and estradiol precipitation after storage at room temperature and after stress conditions. Specifically, the solubility of progesterone and estradiol in the oral pharmaceutical composition of the invention remains after 3 months when stored at room temperature and 60% RH and when storage under stress conditions at 40° C. and 75% RH. Furthermore, estradiol does not precipitate for at least 3 months when stored at room temperature and 60% RH and when storage under stress conditions at 40° C. and 75% RH (See Example 4, Tables 7 and 8).

Furthermore, no presence of Impurity-M under storage conditions has been detected. The Impurity M (i.e. (17α)-pregn-4-ene-3,20-dione) is a degradant impurity of progesterone.

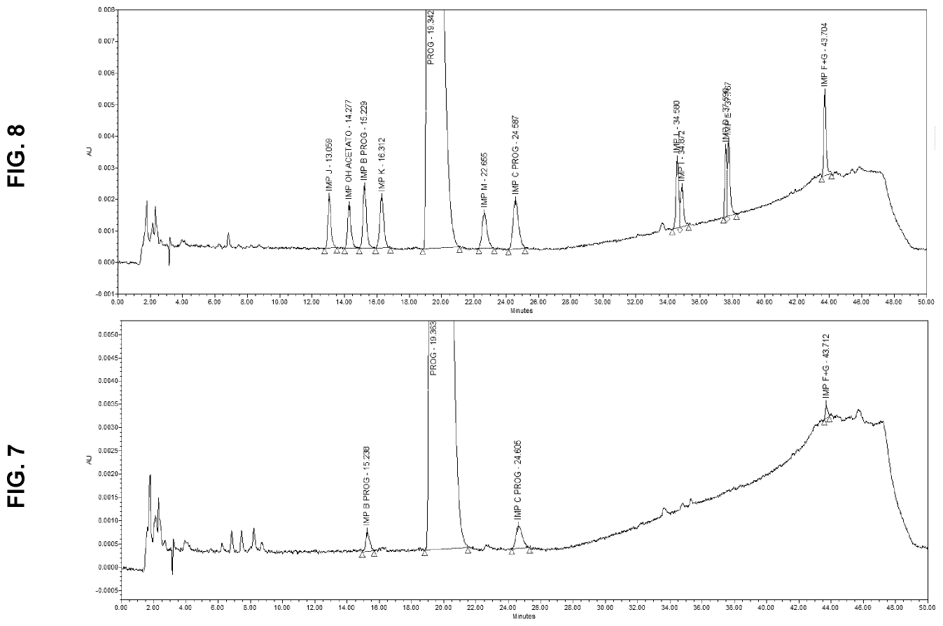

Advantageously, the stable pharmaceutical composition of the present invention comprising progesterone or a pharmaceutically acceptable salt thereof in combination with estradiol or a pharmaceutically acceptable salt thereof, long-chain oil, surfactant, co-surfactant and co-solvent prevents degradation of progesterone to form Impurity-M when stored for at least 3 months at 40° C./75% relative humidity (RH) condition exhibits no presence of Impurity-M as measured by HPLC (See Example 5, FIGS. 7 to 10).

In a further aspect, the invention relates to a stable pharmaceutical composition comprising capsule dosage form suitable for oral administration according to the invention which shows similar and/or improved bioavailability, and furthermore it is bioequivalent to the marketed approved product RLD BIJUVA (See Example 6).

In a further aspect thereof, the invention relates to a novel process for preparing the stable pharmaceutical compositions disclosed herein. The process is capable of preparing the stable pharmaceutical compositions by using room temperature or optionally mild temperature conditions. Unexpectedly, the new temperature conditions do not result in a lower solubility of both APIs. Furthermore, and advantageously, the process for preparing said stable pharmaceutical compositions is more cost-effective, environmentally friendly and energetically sustainable.

The process for preparing stable pharmaceutical composition comprising a capsule dosage form suitable for oral administration, the composition including progesterone or a pharmaceutically acceptable salt thereof, estradiol or a pharmaceutically acceptable salt thereof, a long-chain oil, a surfactant, a co-surfactant and a co-solvent, comprises the steps of:
i) preparing a pre-concentrate of estradiol by suspending the estradiol or a pharmaceutically acceptable salt thereof in a part of the whole long-chain oil and the all of the co-solvent at room temperature;
ii) separately, mixing the rest of the long-chain oil with the surfactant at room temperature, optionally pre-heated at a temperature equal to or lower than 35° C., and the co-surfactant under agitation until homogenization to provide a homogenized mixture;
iii) adding the prepared pre-concentrate of estradiol of step i) into the homogenized mixture of step ii) under agitation until complete solubilisation;
iv) adding progesterone or a pharmaceutically acceptable salt thereof to the solubilized estradiol solution and mixing until homogenous suspension is obtained, and
v) optionally, encapsulating the homogeneous suspension into the capsule dosage form.

The authors of the present invention have found that the preparation of a pre-concentrate of the estradiol or a pharmaceutically acceptable salt thereof as described herein allows reducing the temperature conditions at the process preparation of the pharmaceutical composition of the invention. Furthermore, this new strategy does not impair the solubility of the active ingredients in the pharmaceutical composition thus obtained.

In the process of this aspect of the invention, a pre-concentrate of estradiol or of a pharmaceutically acceptable salt thereof is prepared. The pre-concentrate is prepared by suspending estradiol or a pharmaceutically acceptable salt thereof in a part of the whole long-chain oil and all the co-solvent.

The new strategy of preparing a pre-concentrate with a part of the whole long-chain oil allows the process to be performed at room temperature or at least at mild temperature conditions compared with the prior art which makes such process more cost-effective, environmentally friendly and energetically sustainable.

Furthermore, the estradiol pre-concentrate favors estradiol be easy dissolved, and furthermore without heating or using mild temperature conditions.

Advantageously, the process for preparing the stable pharmaceutical composition can be performed at room temperature or using mild temperature conditions which provides an improved process when compared with Bijuva process (40° C.-65° C.) or with Slayback process (40° C.).

Preferably, the amount of long-chain oil is divided in two parts. A part of about 15-35% of the long-chain oil is used for preparing the pre-concentrate, and the rest up to 100% (65-85%) is used for, separately, mixing the rest of long-chain oil, with the surfactant, optionally pre-heated, and the co-surfactant. It is part of the general knowledge of a person skilled in the art to divide the amount of the long chain oil into another different proportion, so that both are suitable for preparing the pre-concentrate with a first portion and for mixing with the surfactant and the co-surfactant with the second portion.

In an embodiment, the surfactant is liquid at room temperature. In this embodiment, the process is preferable followed at room temperature (See Example 1).

In another embodiment, the surfactant is solid at room temperature. In this embodiment, the following steps use mild temperature conditions (See Example 2).

As used herein, the term “mild temperature” is understood a temperature equal to or lower than 35° C. Preferably, mild temperature is a temperature equal to or lower than 32° C.

Thus, if the surfactant is solid at room temperature, it is pre-heated to a temperature equal to or lower than 35° C., preferable equal to or lower than 32° C., then mixing with the rest up to 100% of the long-chain oil, and the 100% of the co-surfactant under agitation until homogenization to provide a homogenized mixture.

Then, the prepared pre-concentrated of estradiol is added under agitation to the homogenized mixture which is at the same pre-heated surfactant temperature until complete solubilisation of the estradiol pre-concentrated.

Lastly, progesterone or a pharmaceutically acceptable salt thereof is immediately added, and mixed until homogeneous suspension is obtained.

Optionally, encapsulating the homogeneous suspension into a pharmaceutically acceptable capsule.

Surprisingly, the obtained compositions, encapsulated or not, show improved solubility of both active ingredients. The solubility of both active ingredients is not affected by the new temperature conditions, nor by the new prepared pre-concentrated of estradiol. Furthermore, the presence of Impurity-M has not been detected in none of the embodiments (see Example 5, FIGS. 7 to 10).

In still another aspect, the invention relates to a stable pharmaceutical composition comprising a capsule dosage form suitable for oral administration obtainable by the process described above, wherein the composition exhibits no presence of Impurity-M when stored for at least 3 moths at 40° C./75% relative humidity (RH) condition.

In its further aspect, the invention relates to a stable pharmaceutical composition comprising a capsule dosage form suitable for oral administration for use in the treatment of moderate to severe vasomotor symptoms (VMS) related to menopause, in the treatment of secondary amenorrhea, and/or in the prevention of endometrial hyperplasia in non-hysterectomized postmenopausal women who are receiving conjugated estrogen.

### EXAMPLES

The following examples are exemplary and not intended to be limiting. The present disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Example 1—Pharmaceutical Composition and Process for Preparing Thereof

Composition for estradiol hemihydrate and progesterone with the self-emulsifying system Maisine/Labrasol/Capryol is included in Table 1 below:

TABLE 1

| | Quantity/ | Self-emulsifying | % (wt/ |
|---|---|---|---|
| Composition | Batch (g) | system (EG % wt) | total wt) |
| Progesterone Micronized | 1666.7 | | 33.333 |
| Estradiol Hemihydrate | 17.2 | | 0.343 |
| Maisine CC | 2286.1 | 70.00 | 45.720 |
| Labrasol | 490.0 | 15.00 | 9.800 |
| Capryol 90 | 490.0 | 15.00 | 9.800 |
| Ethanol Absolute | 50.0 | | 1.000 |
| Total | 5,000.0 | 100% | 100% |

### —Process Steps—

Firstly, a pre-concentrate was prepared by suspending 17.2 g of estradiol in 450.0 g of Maisine CC and 50.0 g of Ethanol absolute, at room temperature. Then, 1786.1 g Maisine, 490.0 g Labrasol and 490.0 g Capryol 90 were mix under agitation at room temperature. Once the mixture was homogenized the pre-concentrated was added, and optionally the container may be cleaned with 50.0 g of Maisine CC reserved for this purpose, and then mix under agitation until the complete solubilization is obtained. Immediately after the mixture was homogenized, 1666.7 g of Progesterone was added and mix until homogeneous suspension is obtained. Then, the homogeneous suspension is encapsulated into a pharmaceutically acceptable capsule.

### Example 2—Pharmaceutical Composition and Process for Preparing Thereof

Composition for estradiol hemihydrate and progesterone with the self-emulsifying system Maisine/Kolliphor/Plurol Oleique is included in Table 2 below:

TABLE 2

| | Quantity/ | Self-emulsifying | % (wt/ |
|---|---|---|---|
| Composition | Batch (g) | system (E₂ % wt) | total wt) |
| Progesterone Micronized | 1666.7 | | 33.333 |
| Estradiol Hemihydrate | 17.2 | | 0.343 |
| Maisine CC | 1633.0 | 50.00 | 32.660 |
| Kolliphor RH40 | 325.0 | 10.00 | 6.500 |
| Plurol Oleique CC | 1308.1 | 40.00 | 26.160 |
| Ethanol Absolute | 50.0 | | 1.000 |
| Total | 5,000.0 | 100% | 100% |

—Process Steps—

Firstly, a pre-concentrate was prepared by suspending 17.2 g of estradiol in 450.0 g of Maisine CC and 50.0 g of Ethanol absolute, at room temperature. 325.0 g of Kolliphor RH40 is preheated at 32±2° C. before the dispensing. Then, 1133.0 g of the Maisine CC, the 325.0 g of Kolliphor RH40 and 1308.1 g of Plurol Oleique CC are mix under agitation at 32±2° C. Once the mixture is homogenized the pre-concentrated is added, and optionally the container may be cleaned with 50.0 g of Maisine CC reserved for this purpose, and mix under agitation at 32±2° C. Immediately after the mixture is homogenized the progesterone is added and mix until homogeneous suspension is obtained. Then, the homogeneous suspension is encapsulated into a pharmaceutically acceptable capsule.

### Example 3—Solubility and Dissolution Comparison of Compositions with/without Co-Surfactant

### Example 3.1

### —Without Co-Surfactant—

Formulation compositions for estradiol hemihydrate and progesterone including a long chain oil, a surfactant but not including a co-surfactant were solubility tested. Progesterone and estradiol capsules comprising Maisine CC as long chain oil and Kolliphor RH40 as surfactant are prepared following the process of example 2, but without the addition of co-surfactant. Such compositions of progesterone and estradiol are shown in Table 3. Solubilized amount of estradiol and progesterone in suspension determined by HPLC assay method is also included in Table 3.

TABLE 3
Compositions

| without co- | Formulation batch number | | |
|---|---|---|---|
| surfactant (%) | ESPR-0437-42 | ESPR-0437-46-1 | ESPR-0437-46-2 |
| Progesterone | 33.333% | | |
| Micronized | | | |
| Estradiol | 0.343% | | |
| Hemihydrate | | | |
| Maisine CC | 60.4% | 63.0% | 64.3% |
| Kolliphor RH40 | 4.9% | 2.3% | 1.0% |
| — | — | — | — |

| | |
|---|---|
| Ethanol Absolute | 1.0% |
| Total | 100.0% |

Solubility results (Table 3)

| | | | |
|---|---|---|---|
| EH Solubility | 5.03 | 4.88 | 4.86 |
| (mg/g) | | | |
| EH Solubility (%) | 97.5% | 94.4% | 94.0% |
| PROG Solubility | 50.3 | 54.4 | 54.9 |
| (mg/g) | | | |
| PROG Solubility | 10.0% | 10.8% | 10.9% |
| (%) | | | |

The solubility of the estradiol in these compositions is closed to 100%, while the progesterone is solubilized in about 10%.

Dissolution profiles are depicted in FIGS. 1 and 2. FIGS. 1 and 2 show the dissolution profiles of estradiol and progesterone, respectively, for the three formulations without co-surfactant of Table 3. The three formulations presented higher dissolution than Bijuva, being the profiles for the 3 formulations quite similar, for both APIs.

### —With Co-Surfactant—

Compositions for estradiol hemihydrate and progesterone including a long chain oil, a surfactant and further including a co-surfactant were solubility tested. In order to study the effect of the co-surfactant on these systems, progesterone and estradiol capsules comprising Maisine CC as long chain oil, Kolliphor RH40 as surfactant and Plurol Oleique as co-surfactant are prepared following the process of example 2. Compositions of progesterone and estradiol are shown in Table 4. Solubilized amount of estradiol and progesterone in suspension determined by HPLC assay method is also included below in Table 4.

TABLE 4

| | Formulation batch number | | | | | | | |
|---|---|---|---|---|---|---|---|---|
| Composition (%) | | | | | | | ESPR-0437-53-2 | |
| (Self-emulsion | ESPR-0437-50-1 | | ESPR-0437-50-2 | | ESPR-0437-53-1 | | (Ex. 2) | |
| system E₂) | Comparative examples | | | | Invention (E₂) | | | |
| Progesterone Micronized | 33.333% | | | | 33.333% | | | |
| Estradiol Hemihydrate | 0.343% | | | | 0.343% | | | |
| Self-emulsifying system | | (% wt) | | (% wt) | | E₂ | | E₂ |
| | | | | | | (% wt) | | (% wt) |
| Maisine CC | 52.3% | 80.0% | 54.7% | 83.77% | 32.7% | 50.0% | 32.7% | 50.0% |
| Kolliphor RH40 | 6.5% | 10.0% | 4.9% | 7.50% | 9.8% | 15.0% | 6.5% | 10.0% |
| Plurol Oleique CC | 6.5% | 10.0% | 5.7% | 8.73% | 22.9% | 35.0% | 26.1% | 40.0% |
| Total system | | 100% | | 100% | | 100% | | 100% |

| | | |
|---|---|---|
| Ethanol Absolute | 1.0% | 1.0% |
| Total | 100% | 100% |

Solubility results (Table 4)

| | | | | |
|---|---|---|---|---|
| EH Solubility (mg/g) | 4.73 | 4.71 | 4.88 | 4.81 |
| EH Solubility (%) | 91.4% | 91.1% | 94.3% | 93.0% |
| PROG Solubility (mg/g) | 47.1 | 48.4 | 39.0 | 40.4 |
| PROG Solubility (%) | 9.4% | 9.6% | 7.8% | 8.0% |

According to the results obtained, the solubilities of progesterone and estradiol are not adversely affected by the presence or absence of co-surfactant (comparative solubility can be extracted from comparative solubility results of Table 3 versus Table 4).

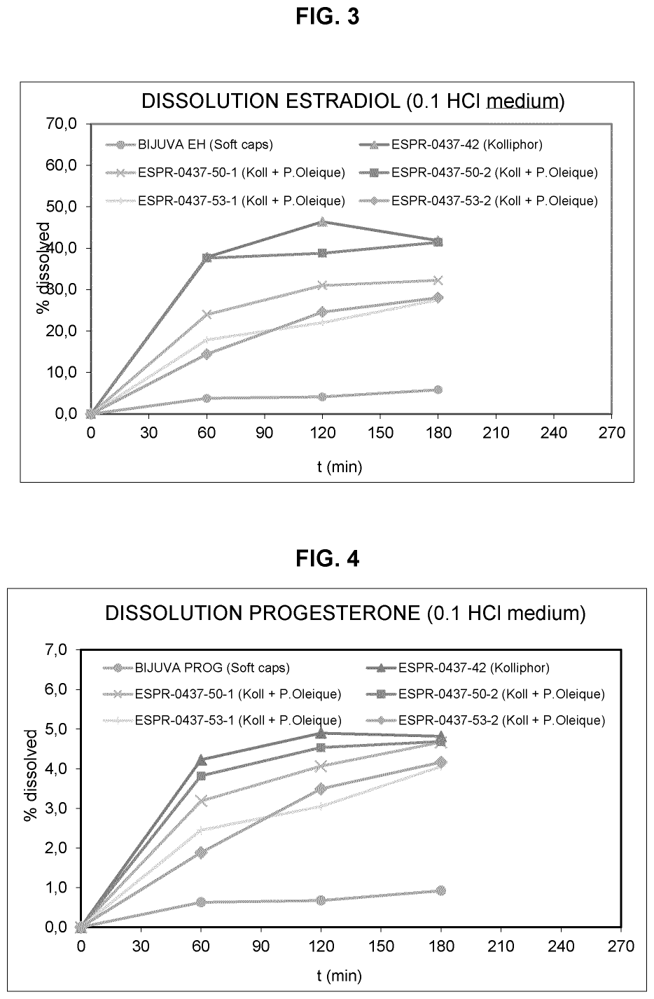

Dissolution profiles are depicted in FIGS. 3 and 4. FIGS. 3 and 4 show the dissolution profiles of estradiol and progesterone, respectively, for the formulations including co-surfactant. The 4 formulations (Table 4) containing co-surfactant are compared against RLD Bijuva and also are compared against the trial ESPR-0437-042 (Table 3, without co-surfactant) and the profiles of the 2 formulations (Table 4, Invention (E₂)) are improved and closer to the RLD Bijuva profiles. From the comparative results, the self-emulsion system E₂ of the invention having higher proportion of Plurol Oleique (co-surfactant) is closer to RLD Bijuva. As a conclusion, the addition of the co-surfactant in the composition promotes the reduction of dissolved amount, however the dissolution profile tends to equal Bijuva profile.

### Example 3.2

In the same manner, Progesterone and estradiol capsules comprising Maisine CC as long chain oil, Labrasol as surfactant and Capryol 90 as co-surfactant are prepared according to example 1 above. Compositions for the self-emulsifying system E₁ are shown in Table 5 below, and compositions for the self-emulsifying system EG are shown in Table 6 below. Solubilized amount of estradiol and progesterone in suspension determined by HPLC assay method is also included in Tables 5 and 6.

TABLE 5

| Composition (%) | | | | | | |
|---|---|---|---|---|---|---|
| (Self-emulsion | Formulation batch number | | | | | |
| system E₁) | ESPR-0437-27-1 | | ESPR-0437-27-2 | | ESPR-0437-27-3 | |
| Progesterone Micronized | 33.333% | | 33.333% | | 33.333% | |
| Estradiol Hemihydrate | 0.343% | | 0.343% | | 0.343% | |
| Self-emulsifying system | | E₁(% wt) | | E₁(% wt) | | E₁ (% wt) |
| Maisine CC | 5.88% | 9.0% | 26.13% | 40.0% | 19.60% | 30.0% |
| Labrasol | 38.53% | 59.0% | 32.66% | 50.0% | 32.66% | 50.0% |
| Capryol 90 | 20.90% | 32.0% | 6.53% | 10.0% | 13.06% | 20.0% |
| Total system E₁ | | 100% | | 100% | | 100% |

| Ethanol Absolute | 1.0% | 1.0% | 1.0% |
|---|---|---|---|
| Total | 100% | | |
| | Solubility results (Table 5) | | |
| EH Solubility (mg/g) | 4.85 | 4.87 | 4.68 |
| EH Solubility (%) | 93.8% | 90.0% | 90.5% |
| PROG Solubility (mg/g) | 55.5 | 49.0 | 49.8 |
| PROG Solubility (%) | 11.0% | 9.3% | 9.9% |

TABLE 6

| Composition (%) | | | | | | |
|---|---|---|---|---|---|---|
| (Self-emulsion | Formulation batch number | | | | | |
| system EG) | ESPR-0437-47-1 | | ESPR-0437-47-2 | | (Ex. 1) | |
| Progesterone Micronized | 33.333% | | 33.333% | | 33.333% | |
| Estradiol Hemihydrate | 0.343% | | 0.343% | | 0.343% | |
| Self-emulsifying system | | EG(% wt) | | EG(% wt) | | EG(% wt) |
| Maisine CC | 35.93% | 55.0% | 39.19% | 60.0% | 45.70% | 70.0% |
| Labrasol | 26.13% | 40.0% | 13.06% | 20.0% | 9.80% | 15.0% |
| Capryol 90 | 3.26% | 5.0% | 13.06% | 20.0% | 9.80% | 15.0% |
| Total system EG | | 100% | | 100% | | 100% |

| Ethanol Absolute | 1.0% | 1.0% | 1.0% |
|---|---|---|---|
| Total | 100% | | |
| | Solubility results (Table 6) | | |
| EH Solubility (mg/g) | 4.84 | 4.80 | 4.70 |
| EH Solubility (%) | 93.5% | 94.6% | 91.0% |
| PROG Solubility (mg/g) | 45.3 | 60.1 | 59.5 |
| PROG Solubility (%) | 9.0% | 12.0% | 11.8% |

The different amount of the excipients used, and so that, the ratio among them, didn't show remarkable differences in the solubility of estradiol and progesterone in the different systems (Table 5 and Table 6), being able to solubilize about 90 to 95% of estradiol, and 9 to 12% of progesterone.

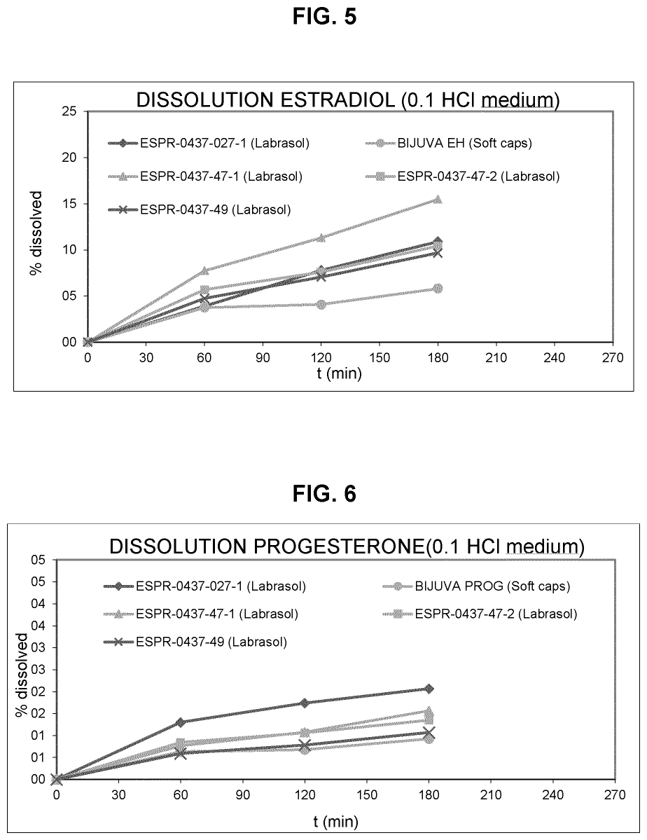

Dissolution profiles are depicted in FIGS. 5 and 6. FIGS. 5 and 6 show the dissolution profiles of estradiol and progesterone, respectively, for the formulations of Tables 5 and 6 above. The shown dissolution profiles are in line with the results obtained in previous examples wherein Plurol Oleique as co-surfactant was used when compared with RLD Bijuva dissolution profiles. Therefore, it is preferable the presence of higher amounts of co-surfactant which results in a decrease of the dissolution profiles for both APIs, and thus results in a more similar profile when compared with RLD Bijuva profile. (See FIGS. 5 and 6).

### —Conclusions—

Based on the results obtained from the systems of the example 3 it could be concluded that:
The solubility of estradiol and progesterone is maintained in every system when varying the presence or absence of co-surfactant.
The dissolution profiles are impacted by selection of surfactant and co-surfactant.
The increase of the co-surfactant amount in the system provides lower dissolution profiles more similar to RLD Bijuva.

### Example 4—Study Stability on Storage

This was a study performed on the composition prepared according to the example 1, based on Table 1, and furthermore, on the composition prepared according to the example 2, based on Table 2.

### —Stability Study: Composition Obtainable According to Example 1—

Table 7 below shows the % of progesterone and estradiol that is dissolved initially and after 1 month and 3 months at different temperatures and RH conditions. Two batches LFD0817 and LFD0818 manufactured according to the example 1 are put on stability and results are shown below:

TABLE 7

| | PROGESTERONE | ESTRADIOL |
|---|---|---|
| DESCRIPCION | % dissolved | % dissolved |
| Maisine CC (45.7%); Labrasol | 12% | 91% |
| (9.8%); Capryol 90 (9.8%); | | |
| Ethanol (1.0%) (Ex. 1/Table 1) | | |
| Batch number LFD0817 (1 month) | | |
| Estradiol/Progesterone, stored at | 13% | 95% |
| 25° C./60% RH for 1 M | | |
| Estradiol/Progesterone, stored at | 13% | 95% |
| 30° C./65% RH for 1 M | | |
| Estradiol/Progesterone, stored at | 13% | 95% |
| 40° C./75% RH for 1 M | | |
| Batch number LFD0817 (3 | | |
| months) | | |
| Estradiol/Progesterone, stored at | 12% | 96% |
| 25° C./60% RH for 3 M | | |
| Estradiol/Progesterone, stored at | 12% | 97% |
| 30° C./65% RH for 3 M | | |
| Estradiol/Progesterone, stored at | 13% | 97% |
| 40° C./75% RH for 3 M | | |
| Batch number LFD0818 (1 month) | | |
| Estradiol/Progesterone, stored at | 13% | 94% |
| 25° C./60% RH for 1 M | | |
| Estradiol/Progesterone, stored at | 14% | 95% |
| 30° C./65% RH for 1 M | | |
| Estradiol/Progesterone, stored at | 14% | 94% |
| 40° C./75% RH for 1 M | | |
| Batch number LFD0818 (3 | | |
| months) | | |
| Estradiol/Progesterone, stored at | 13% | 99% |
| 25° C./60% RH for 3 M | | |
| Estradiol/Progesterone, stored at | 13% | 100% |
| 30° C./65% RH for 3 M | | |
| Estradiol/Progesterone, stored at | 13% | 97% |
| 40° C./75% RH for 3 M | | |

—Stability Study. Composition Obtainable According to Example 2—

This was a study performed on the composition manufactured according to the example 2. The formulation composition is based on Table 2.

For the stability tests, the same methodology and conditions were used as detailed above for the formulation composition based on Table 1.

Table 8 below shows the % of progesterone and estradiol that is dissolved initially and after 1 month and 3 months at different temperatures and RH conditions. Two batches LFD0824 and LFD0825 manufactured according to the example 2 are put on stability and results are shown below:

TABLE 8

| | PROGESTERONE | ESTRADIOL |
|---|---|---|
| DESCRIPTION | % dissolved | % dissolved |
| Maisine (32.6%); Kolliphor RH40 | 8% | 93% |
| (6.5%); Plurol Oleic (26.1%) | | |
| (Ex. 2/Table 2) | | |
| Batch number LFD0824 (1 month) | | |
| Estradiol/Progesterone, stored at | 9% | 97% |
| 25° C./60% RH for 1 M | | |
| Estradiol/Progesterone, stored at | 10% | 99% |
| 30° C./65% RH for 1 M | | |
| Estradiol/Progesterone, stored at | 10% | 98% |
| 40° C./75% RH for 1 M | | |
| Batch number LFD0824 (3 | | |
| months) | | |
| Estradiol/Progesterone, stored at | 8% | 100% |
| 25° C./60% RH for 3 M | | |
| Estradiol/Progesterone, stored at | 8% | 99% |
| 30° C./65% RH for 3 M | | |
| Estradiol/Progesterone, stored at | 8% | 98% |
| 40° C./75% RH for 3 M | | |
| Batch number LFD0825 (1 month) | | |
| Estradiol/Progesterone, stored at | 8% | 98% |
| 25° C./60% RH for 1 M | | |
| Estradiol/Progesterone, stored at | 9% | 100% |
| 30° C./65% RH for 1 M | | |
| Estradiol/Progesterone, stored at | 8% | 97% |
| 40° C./75% RH for 1 M | | |
| Batch number LFD0825 (3 | | |
| months) | | |
| Estradiol/Progesterone, stored at | 8% | 100% |
| 25° C./60% RH for 3 M | | |
| Estradiol/Progesterone, stored at | 8% | 100% |
| 30° C./65% RH for 3 M | | |
| Estradiol/Progesterone, stored at | 8% | 100% |
| 40° C./75% RH for 3 M | | |

### —Stability Study Conclusions:

Based on the results above, both APIs, progesterone and estradiol, remain dissolved at the same percentages as initial (Table 7 and 8) and no precipitation is thus produced at least after 3 months at RT, intermediate conditions of 30° C. and 65% RH and in stress conditions of 40° C. and 75% RH.

### Example 5—Progesterone Related Substances

### Equipment and Materials

Liquid Chromatography HPLC equipped with degasser, pump, automatic injector, oven and UV detector.
Analytical balance.
Column: Poroshell 120 EC-C18 4.6 mm×250 mm×4 μm
Centrifuge
Heated stirring plate
Ultrasonic bath

### Reagents and Standards

HPLC grade purified water
HPLC grade methanol
HPLC grade Acetonitrile
Progesterone Standard
Pancreatin from porcine pancreas
Tetrahydrofuran (THF)
Placebo
Diluent: AcN/H₂O (50:50 v/v)

Procedure (Flasks of different volumes, weights, and dilutions other than those described can be used as long as the final concentrations are maintained.)
Porcine Pancreas Pancreatin Solution Preparation (0.2% w/v)

Weigh out approximately 200 mg of pancreatin and transfer to a glass with 100 mL of water. Stir magnetically for 15 minutes at approximately 200 rpm.

Note: Prepare sufficient quantity according to number of samples. Prepare this solution daily.
Impurities Standard Solution Preparation (0.2%)
Progesterone Stock Solution: Weigh exactly 10 mg standard Progesterone into a 50.0 mL flask. Add 25 mL of AcN and sonicate for 10 minutes or until complete dissolution. Allow to warm and bring up to volume with AcN (concentration 200 μg/mL).
Working standard solution of 0.2% Progesterone: Take 1.0 mL of the Progesterone stock solution and transfer to a 250.0 mL flask. Bring up to volume with diluent. (Concentration 0.8 μg/mL of Progesterone).
Placebo preparation
Placebo Stock Solution: Weigh about 1000 mg of placebo fill into a 250.0 mL flask. Add approximately 35 mL of Pancreatin solution in water at 0.2% and maintain heated magnetic stirring at approximately 150 rpm and 40° C. for 30 minutes. After this time, add 60 mL of THF and continue stirring on the heated stirrer plate for 15 minutes. After that, add approximately 100 mL of MeOH and keep stirring on a heated stirrer plate (increase to 200 rpm to facilitate solubilization of the filling) for 30 minutes. Remove the magnetic stirrer by washing it with MeOH. Sonicate the flasks for 10 minutes. Once tempered, bring up to volume with MeOH. Vortex the flasks before the final dilution of the placebo.
Final dilution of the placebo: Take 2.0 mL of the placebo stock solution and transfer to a 10.0 mL flask. Bring up to volume with diluent. Vortex and centrifuge an aliquot at 4000 rpm for 3 minutes. Take the supernatant and vitalize.
Sample preparation
Progesterone Stock solution: weight 5 sample capsules and place into a 250.0 ML flask. Add approximately 35 mL of Pancreatin solution in water at 0.2% and maintain under heated magnetic stirring for approximately 150 rpm and 40° C. for 30 minutes. After that, add 60 mL of THF and keep stirring in heated stirrer plate (increase at 200 rpm to facilitate solubilization of the filling) for 30 minutes. Remove the magnetic stirring by washing it with MeOH. Sonicate the flasks for 100 minutes. Once tempered, bring up to volume with MeOH. Vortex the flasks before the final sample dilution (Concentration 2000 μg/mL of progesterone).
Final sample dilution: Take 2.0 mL of the stock solution and transfer to a 10.0 mL flask. Bring up to volume with diluent. Vortex and centrifuge an aliquot at 4000 rpm for 3 minutes. Take the supernatant and vitalize (concentration 400 μg/mL of progesterone).
Chromatographic conditions
Column: Poroshell 120 EC-C18 4.6 mm×250 mm (4 μm)
Flow: 1.0 mL/min
Gradient mobile phase:

| Time | H₂O (A) | MeOH/AcN (50/50 v/v) (B) |
|---|---|---|
| 0 | 40 | 60 |
| 24 | 40 | 60 |
| 40 | 10 | 90 |
| 44 | 10 | 90 |
| 46 | 40 | 60 |
| 50 | 40 | 60 |

Injector temperature: 25° C.
Column temperature: 40° C.
Injection volume: 10 μL
Chromatogram duration: 50 min
Detector: UV 242 nm BW 4 nm Ref. 350/4 nm
Example of injection sequence
1 diluent injection
1 placebo injection
6 working STD injections
1 injection of each sample (5 sample injections between 2 STD injections)
1 working STD injection
1 diluent injection
System Suitability

The system works properly if:
N≥2000 for Progesterone
0.8≤Tailing≤1.5 for the main Progesterone peak.
RSD≤5.0% of the 6 injections of the standard (STD1).
Report Limit value

| | Report limit |
|---|---|
| Progesterone | >0.1% |
| | 0.4 μg/mL |

### Calculations

Versus Impurities standard

$$\% I = \frac{{A}_{I}}{{A}_{std}} \times \frac{{P}_{std}}{{D}_{std}} \times {D}_{m} \times \frac{1}{N} \times \frac{1}{LC} \times F \times 100 \%$$

Where:
AI: Area of impurity peak in sample
Astd: mean of the impurity areas in the impurity standard solution at 0.2%
Pstd: weight of the impurity standard in the impurity standard solution at 0.2%
Dstd: dilution of the standard in the impurity standard solution at 0.2% (mL)
Dm: sample dilution (mL).
LC: API dose per capsule (mg)
N: number of capsules
F: Progesterone purity in standard solution (per one unit).

### —Evaluation of Impurity M—

Evaluation of Impurities formed in the formulation compositions of Examples 1 and 2, when stored at 40° C. and 75% HR for three months, were conducted, and the results included in Table 9.1 and 9.2 below, respectively.

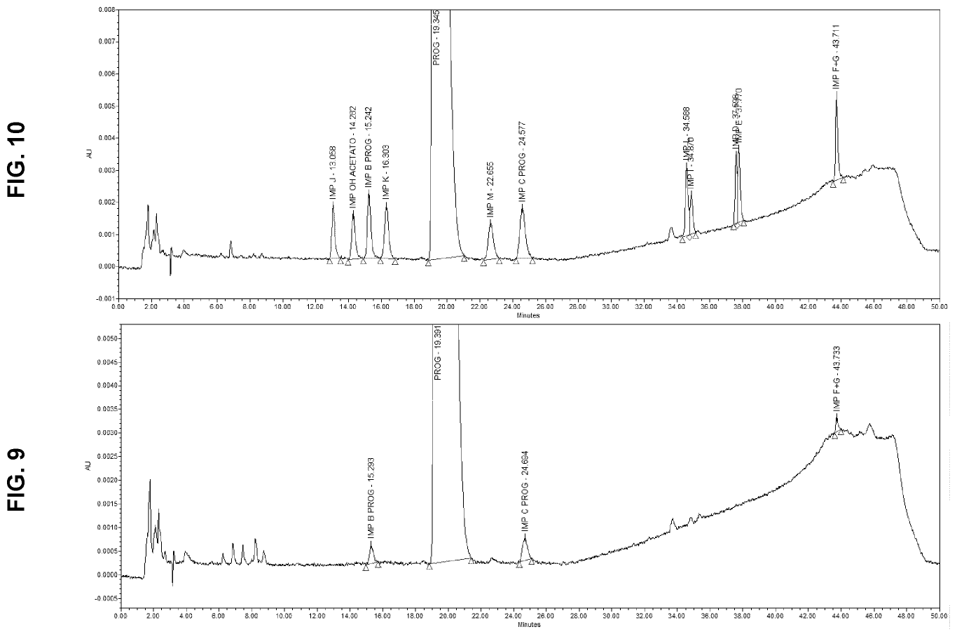

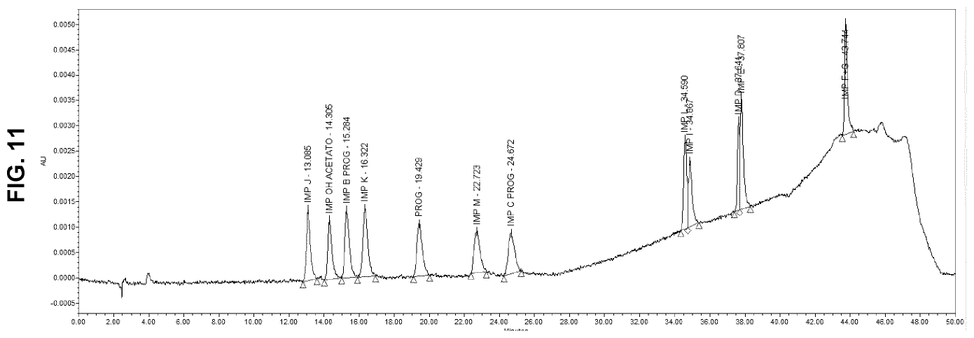

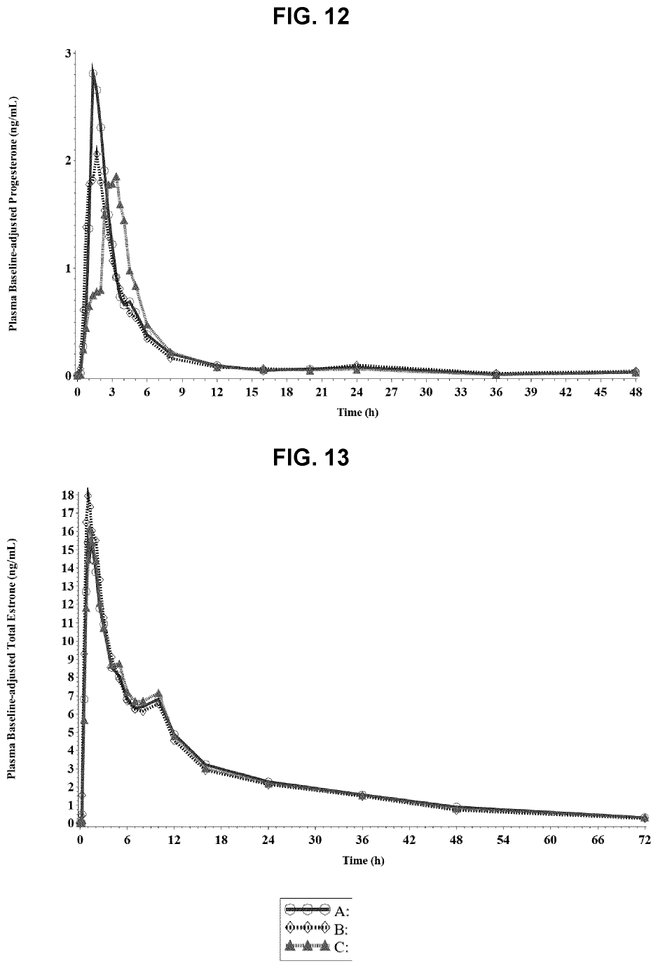

Chromatograms depicted in FIGS. 7 and 9, and compared with chromatograms depicted in FIGS. 8 and 10 reveal the no presence of Impurity M in the dosage forms of the invention. FIG. 11, chromatogram of Progesterone impurities, shows Impurity M signal at 22.723.

Particularly, comparison of FIGS. 7 and 8 demonstrate that in the sample solution of Labrasol formulation (from Example 1) no presence of Impurity M is detected (Table 9.1).

Particularly, comparison of FIGS. 9 and 10 demonstrate that in the sample solution of Kolliphor formulation (from Example 2) no presence of Impurity M is detected (Table 9.2).

TABLE 9.1
Related substance data of progesterone

| | | | | impurities (%) | | | |
|---|---|---|---|---|---|---|---|
| | | | | LFD0817B | | LFD0818B | |
| | | | Specification | | 40° C./ | | 40° C./ |
| | Name of the | | (as per ICH) | | 75% RH | | 75% RH |
| S. No. | impurity | RRT | % impurity | initial | 3 months | initial | 3 months |
| 1 | Single max unknown | 0.43 | 0.2 | — | 0.06 | NA | 0.04 |
| 3 | Impurity J | 0.68 | | 0.00 | 0.00 | 0.00 | 0.00 |
| 4 | Impurity OH-Prog-Ac | 0.74 | | 0.00 | 0.00 | 0.00 | 0.00 |
| 5 | Impurity B | 0.79 | | 0.05 | 0.06 | 0.06 | 0.06 |
| 6 | Impurity K | 0.84 | | 0.00 | 0.00 | 0.00 | 0.00 |
| 7 | Impurity H (288 nm) | 0.84 | | 0.00 | 0.00 | 0.00 | 0.00 |
| 8 | Impurity M | 1.17 | | 0.00 | 0.00 | 0.00 | 0.00 |
| 9 | Impurity C | 1.27 | | 0.12 | 0.11 | 0.11 | 0.11 |
| 10 | Impurity L | 1.79 | | 0.00 | 0.00 | 0.00 | 0.00 |
| 11 | Impurity I | 1.80 | | 0.00 | 0.00 | 0.00 | 0.00 |
| 12 | Impurity D | 1.94 | | 0.00 | 0.00 | 0.00 | 0.00 |
| 13 | Impurity E | 1.95 | | 0.00 | 0.00 | 0.00 | 0.00 |
| 14 | Impurity F + G | 2.26 | | 0.00 | 0.00 | 0.00 | 0.00 |
| 15 | Total impurities | NA | 0.6 | NR | NR | NR | NR |

NA: NOT APPLICABLE;
NR: NOT REPORTABLE

TABLE 9.2
Related substance data of progesterone

| | | | | impurities (%) | | | |
|---|---|---|---|---|---|---|---|
| | | | | LFD0824B | | LFD0825B | |
| | | | Specification | | 40° C./ | | 40° C./ |
| | Name of the | | (as per ICH) | | 75% RH | | 75% RH |
| S. No. | impurity | RRT | % impurity | initial | 3 months | initial | 3 months |
| 1 | Single max unknown | 0.43 | 0.2 | — | 0.06 | 0.04 | 0.08 |
| 2 | Impurity J | 0.68 | | 0.00 | 0.00 | 0.00 | 0.00 |
| 3 | Impurity OH-Prog-Ac | 0.74 | | 0.00 | 0.00 | 0.00 | 0.00 |
| 4 | Impurity B | 0.79 | | 0.05 | 0.06 | 0.06 | 0.07 |
| 5 | Impurity K | 0.84 | | 0.00 | 0.00 | 0.00 | 0.00 |
| 6 | Impurity H (288 nm) | 0.84 | | 0.00 | 0.00 | 0.00 | 0.00 |
| 7 | Impurity M | 1.17 | | 0.00 | 0.00 | 0.00 | 0.00 |
| 8 | Impurity C | 1.27 | | 0.12 | 0.11 | 0.11 | 0.14 |
| 9 | Impurity L | 1.79 | | 0.00 | 0.00 | 0.00 | 0.00 |
| 10 | Impurity I | 1.80 | | 0.00 | 0.00 | 0.00 | 0.00 |
| 11 | Impurity D | 1.94 | | 0.00 | 0.00 | 0.00 | 0.00 |
| 12 | Impurity E | 1.95 | | 0.00 | 0.00 | 0.00 | 0.00 |
| 13 | Impurity F + G | 2.26 | | 0.00 | 0.00 | 0.00 | 0.00 |
| 14 | Total impurities | NA | 0.6 | NR | NR | NR | NR |

NA: NOT APPLICABLE;
NR: NOT REPORTABLE

### Example 6: Clinical Study (In Vivo): Comparative Bioavailabilities

### Objectives:

The primary objective of this study was to evaluate the comparative bioavailability between two formulations of Estradiol/Progesterone 1 mg/100 mg capsules, compositional and prepared in accordance with Example 1 and Example 2 of the application, and Bijuva® (Estradiol and Progesterone) 1 mg/100 mg capsules (Marketing Authorization Holder [MAH]: TherapeuticsMD, Inc., USA) after a single dose in healthy postmenopausal (physiologically or surgically) female subjects under fasted conditions.

The secondary objective of this study was to evaluate the safety and tolerability of the study treatments.

### Methodology:

This was an open-label, single-dose, randomized, three-period, three-treatment, three sequence, crossover study.
This study was designed to evaluate the comparative bioavailability of progesterone and total estrone in healthy, postmenopausal (physiologically or surgically) female subjects under fasted conditions, between two (2) test formulations and a reference product.
In each period, subjects were dosed with test product 1, test product 2, or the reference product, in accordance with the randomization scheme, after an overnight fast of at least 10 hours.
Concentrations of progesterone and total estrone were measured from samples collected prior to dosing (at −1, −0.5, and 0 hours) for baseline measurement in each period.
Concentrations of progesterone were measured from the samples collected over a 48-hour interval after dosing in each period.
Concentrations of total estrone were measured from the samples collected over a 72-hour interval after dosing in each period.
The following pharmacokinetic (PK) parameters were estimated for baseline-adjusted and measured progesterone and total estrone using a non-compartmental approach:
Cmax, AUCt, AUCinf, Tmax, Kel, and Thalf.
There was no formal statistical evaluation of safety or tolerability. The assessment of safety and tolerability was based primarily on the incidence, frequency, and severity of adverse events (AEs).

The compositions of the examples of the application (as administered in treatment) were tested.

### Main Criteria for Inclusion:

The study population included non-smoking, postmenopausal (physiologically or surgically) female volunteers 18 years of age or older, with a body mass index (BMI) ≥19 and ≤33 kg/m2, who were judged to be healthy based on a medical history, 12-lead electrocardiogram (ECG), laboratory evaluation (hematology, biochemistry, urinalysis and serology), physical examination, and vital signs measurements (blood pressure, pulse rate, respiration rate, and temperature). Eligible subjects had negative test results for serum human chorionic gonadotropin (in females), urine cotinine, and urine drugs of abuse at screening.

### Duration of Treatment:

A single-dose treatment administered in each of three periods, separated by a washout of 28 days.
Statistical Methods:

Analysis of variance (ANOVA) (PROC GLMfrom SAS® version 9.4) was performed on log-transformed baseline-adjusted plasma progesterone and total estrone, AUCt, AUCinf, and Cmax.

As the study was dosed in groups, the ANOVA was conducted with the following factors: treatment, group, treatment*group, period (group), sequence, sequence*group, and subject (sequence*group) and if the treatment*group interaction was found to be statistically insignificant (p-value ≥0.05) for the primary variables, a new statistical analysis was conducted using the conventional ANOVA model with sequence, period, treatment, and subject within sequence as fixed factors to assess comparative bioavailability.

Based on log-transformed data, ratios of the geometric means for treatments and the corresponding 90% confidence intervals (CIs) were calculated for AUCt, AUCinf, and Cmax for the comparison of treatment A versus treatment C and treatment B versus treatment C.

These contrasts were estimated separately using only the data from the treatments involved in the comparison. The power of the tests for treatment differences were estimated in the ANOVA.

These statistics were used to evaluate the performance of the test formulations in relation to the reference product.

### Pharmacokinetic Results:

Summary of Study Results Based on Baseline-adjusted Plasma Progesterone Levels

Pharmacokinetic Results:
Summary of Study Results Based on Baseline-adjusted Plasma Progesterone Levels
Based on Raw Data

| | | | Arithmetic | | | | 90% | Intra- | |
|---|---|---|---|---|---|---|---|---|---|
| | | | Mean | Geometric | | Ratio | Confidence | Sbj | Power |
| Parameter | Trt | n | (CV %) | Mean | Contrast | (%) | Interval | CV(%) | (%) |
| *AUCt | A | 28 | 9.1241 | 5.4475 | A vs C | 107.17 | 91.69- | 33 | 41.77 |
| (hr*ng/mL) | | | (175) | | | | 125.25 | | |
| | C | 26 | 8.3125 | 5.0833 | | | | | |
| | | | (200) | | | | | | |
| *Cmax | A | 28 | 3.5438 | 1.3991 | A vs C | 116.35 | 97.21- | 38 | 11.87 |
| (ng/mL) | | | (232) | | | | 139.25 | | |
| | C | 26 | 2.3876 | 1.2025 | | | | | |
| | | | (257) | | | | | | |

| | |
|---|---|
| Treatment A | Estradiol/Progesterone 1 mg/100 mg capsules, compositional and prepared in accordance |
| (Test-1) | with Example 1 of the application |
| Treatment C | Bijuva ® (Estradiol and Progesterone) 1 mg/100 mg capsules, Batch No.: 3551122A |
| (Reference) | (TherapeuticsMD, Inc., USA) |

*conventional ANOVA model with sequence, period, treatment, and subject within sequence as fixed factors

The term “Cmax” refers to the maximum value of blood concentration shown on the curve that represents changes in blood concentrations of an active pharmaceutical ingredient (e.g., progesterone or estradiol) overtime.

The term “area under the curve” (“AUC”) refers to the area under the curve defined by changes in the blood concentration of an active pharmaceutical ingredient (e.g., estradiol or progesterone) over time following the administration of a dose of the active pharmaceutical ingredient.

“AUCt” is the area under the concentration-time curve from time zero to time t following the administration of a dose, wherein t is the last time point with a measurable concentration.

“AUC0inf” is the area under the concentration-time curve extrapolated to infinity following the administration of a dose.

CV represents the arithmetic mean.

The Ratio % is measured in relation to the geometric mean and the values resulted from: AUCt: 5.4475/5.0833=1,07017×100=107.17%; Cmax: 1.3991/1.2025=1,1635×100=116.35%

Based on Raw Data

| | | | Arithmetic | Geometric | | Ratio | 90% Confidence | Intra-Sbj | Power |
|---|---|---|---|---|---|---|---|---|---|
| Parameter | Trt | n | Mean (CV %) | Mean | Contrast | (%) | Interval | CV(%) | (%) |
| *AUCt | B | 29 | 8.5812 (127) | 5.7560 | B vs C | 116.36 | 98.33-137.69 | 36 | 14.38 |
| (hr*ng/mL) | C | 26 | 8.3125 (200) | 4.9468 | | | | | |
| *Cmax | B | 29 | 3.0459 (117) | 2.0199 | B vs C | 188.13 | 157.92-224.11 | 37 | <0.01 |
| (ng/mL) | C | 26 | 2.3876 (257) | 1.0737 | | | | | |

| | |
|---|---|
| Treatment B | Estradiol/Progesterone 1 mg/100 mg capsules, compositional and prepared in accordance |
| (Test-2) | with Example 2 of the application |
| Treatment C | Bijuva ® (Estradiol and Progesterone) 1 mg/100 mg capsules, Batch No.: 3551122A |
| (Reference) | (TherapeuticsMD, Inc., USA) |

*conventional ANOVA model with sequence, period, treatment, and subject within sequence as fixed factors

### Summary of Stud Results Based on Baseline-Adjusted Plasma Total Estrone Levels

Pharmacokinetic Results:
Summary of Study Results Based on Baseline-adjusted Plasma Total Estrone Levels
Based on Raw Data

| | | | Arithmetic | | Geometric | | Ratio | 90% Confidence | Intra-Sbj | Power |
|---|---|---|---|---|---|---|---|---|---|---|
| Parameter | Trt | n | Mean (CV %) | | Mean | Contrast | (%) | Interval | CV(%) | (%) |
| *Cmax | A | 28 | 23.665 | (39) | 22.127 | A vs C | 97.99 | 88.99-107.90 | 21 | 95.68 |
| (ng/mL) | C | 28 | 23.430 | (34) | 22.581 | | | | | |
| AUCt | A | 28 | 179.950 | (37) | 158.081 | A vs C | 90.92 | 85.42-96.77 | 10 | 95.74 |
| (hr*ng/mL) | C | 28 | 179.554 | (38) | 173.878 | | | | | |
| AUCinf | A | 28 | 202.609 | (38) | 177.839 | A vs C | 92.48 | 87.42-97.83 | 9 | 99.35 |
| (hr*ng/mL) | C | 28 | 197.582 | (37) | 192.299 | | | | | |

| | |
|---|---|
| Treatment A | Estradiol/Progesterone 1 mg/100 mg capsules, compositional and prepared in accordance |
| (Test-1) | with Example 1 of the application |
| Treatment C | Bijuva ® (Estradiol and Progesterone) 1 mg/100 mg capsules, Batch No.: 3551122A |
| (Reference) | (TherapeuticsMD, Inc., USA) |

*conventional ANOVA model with sequence, period, treatment, and subject within sequence as fixed factors

Based on Raw Data

| | | | Arithmetic | | Geometric | | Ratio | 90% Confidence | Intra-Sbj | Power |
|---|---|---|---|---|---|---|---|---|---|---|
| Parameter | Trt | n | Mean (CV %) | | Mean | Contrast | (%) | Interval | CV(%) | (%) |
| *AUCt | B | 30 | 176.378 | (40) | 160.533 | B vs C | 93.88 | 88.14-99.99 | 14 | 99.26 |
| (hr*ng/mL) | C | 28 | 179.554 | (38) | 170.998 | | | | | |
| *Cmax | B | 30 | 26.101 | (30) | 24.866 | B vs C | 109.89 | 101.70-118.74 | 17 | 86.61 |
| (ng/mL) | C | 28 | 23.430 | (34) | 22.628 | | | | | |
| AUCinf | B | 30 | 194.461 | (38) | 173.902 | B vs C | 90.12 | 84.82-95.75 | 11 | 94.40 |
| (hr*ng/mL) | C | 28 | 197.582 | (37) | 192.968 | | | | | |

| | |
|---|---|
| Treatment B | Estradiol/Progesterone 1 mg/100 mg capsules, compositional and prepared in accordance |
| (Test-2) | with Example 2 of the application |
| Treatment C | Bijuva ® (Estradiol and Progesterone) 1 mg/100 mg capsules, Batch No.: 3551122A |
| (Reference) | (TherapeuticsMD, Inc., USA) |

*conventional ANOVA model with sequence, period, treatment, and subject within sequence as fixed factors

Safety Results:

The administration of the study drugs was well tolerated by the healthy female subjects participating in this study.
Table below summarizes the treatment emergent adverse events (TEAEs) occurring in the study:

| | | | | | | | | Concomitant | |
|---|---|---|---|---|---|---|---|---|---|
| | Severity | | | Relationship to the IMP | | | | Therapy | |
| Trt | Mild | Moderate | Severe | Probable | Possible | Unlikely | Unrelated | DT | NDT |
| A | 2 | 0 | 0 | 0 | 1 | 0 | 1 | 0 | 0 |
| B | 3 | 0 | 0 | 0 | 3 | 0 | 0 | 0 | 1 |
| C | 2 | 0 | 0 | 0 | 2 | 0 | 0 | 0 | 0 |
| Total | 7 | 0 | 0 | 0 | 6 | 0 | 1 | 0 | 1 |

DT, drug therapy; IMP, investigational medicinal product; NDT, non-drug therapy; Trt, treatment.
Treatment A (Test 1): Estradiol/Progesterone 1 mg/100 mg capsules, composition and process as Example 1 of the application;
Treatment B (Test 2): Estradiol/Progesterone 1 mg/100 mg capsules, composition and process as Example 2 of the application;
Treatment C (Reference): Bijuva ® (Estradiol and Progesterone) 1 mg/100 mg capsules, Batch No.: 3551122A (TherapeuticsMD, Inc., USA)

A total of 7 TEAEs were reported by 7 subjects (23.3% of subjects dosed) in the study, of which 6 TEAEs affecting 6 subjects (20.0%) were assessed as treatment related (possible relationship to the investigational medicinal product [IMP]).

No serious adverse events (SAEs) were reported during the conduct of this study. All AEs were mild in severity and resolved prior to the end of the study. No subjects were discontinued by the investigator due to AEs, and none of the AEs had a significant impact on the safety of the subjects or on the integrity of the study results.

The study medications were well tolerated by the healthy volunteers who participated in the study.

### —Conclusions:

Treatment A versus Treatment C

### Baseline-Adjusted Progesterone

The peak and total systemic exposure of baseline-adjusted progesterone were approximately 16% higher and relatively similar, respectively (Cmax ratio=116.35%; AUCt ratio=107.17%) for test product 1 compared to the reference product of Bijuva.

### Baseline-Adjusted Total Estrone

The peak and total systemic exposure of baseline-adjusted total estrone were relatively similar (Cmax ratio=97.99%; AUCt ratio=90.92%; AUCinf ratio=92.48%) for test product 1 compared to the reference product.

Test product 1, Estradiol/Progesterone 1 mg/100 mg capsules, composition and process as Example 1 of the application displayed higher peak systemic exposure and relatively similar total systemic exposure for baseline-adjusted progesterone, and relatively similar peak and total systemic exposure for baseline-adjusted total compared to the reference product, Bijuva® (Estradiol and Progesterone) 1 mg/100 mg capsules, Batch No.: 3551122A (MAH: TherapeuticsMD, Inc., USA), after a single 1 mg estradiol/100 mg progesterone dose in healthy, postmenopausal (physiologically or surgically) female subjects under fasted conditions.

### Treatment B Versus Treatment C

### Baseline-Adjusted Progesterone

The peak and total systemic exposure of baseline-adjusted progesterone were approximately 88% and 16% higher, respectively (Cmax ratio=188.13%; AUCt ratio=116.36%) for test product 2 compared to the reference product.

### Baseline-Adjusted Total Estrone

The peak and total systemic exposure of baseline-adjusted total estrone were relatively similar (Cmax ratio=109.89%; AUCt ratio=93.88%; AUCinf ratio=90.12%) for test product 2 compared to the reference product.

Test product 2, Estradiol/Progesterone 1 mg/100 mg capsules, composition and process as Example 2 of the application displayed higher peak and total systemic exposure for baseline-adjusted progesterone, and relatively similar peak and total systemic exposure for baseline-adjusted total estrone compared to the reference product, Bijuva® (Estradiol and Progesterone) 1 mg/100 mg capsules, Batch No.: 3551122A (MAH: TherapeuticsMD, Inc., USA), after a single 1 mg estradiol/100 mg progesterone dose in healthy, postmenopausal (physiologically or surgically) female subjects under fasted conditions.

Therefore, the results of the clinical assays (in vivo) reveal that the stable pharmaceutical compositions of the invention have a bioavailability similar and/or improved compared to Bijuva composition.

1.-24. (canceled)

25. A stable pharmaceutical composition comprising a capsule dosage form suitable for oral administration, wherein the composition comprises:
two active ingredients that are
progesterone or a pharmaceutically acceptable salt thereof, and
estradiol or a pharmaceutically acceptable salt thereof;
a self-emulsifying system that comprises
a long-chain oil selected from the group consisting of Grapeseed oil, Mixture of refined soybean oil, glyceryl distearate and polyglyceryl-3 dioleate, Wheat germ oil, Cottonseed oil, Rice bran oil, Glyceryl monolinoleate, Soybean oil, Glyceryl monooleate, and Olive oil;
a surfactant selected from the group consisting of Linoleoyl Polyoxyl-6 glycerides, Capryolcaproyl polyoxyl-8-glycerides, Polysorbate 80, polyoxyl-40 hydrogenated castor oil, Oleoyl polyoxyl-6 glycerides, Polyoxyl-35 castor oil, and Polyoxyl-15 Hydrostearate; and
a co-surfactant selected from the group consisting of propylene glycol monocaprylate, Propylene glycol monolaurate, and polyglyceryl-3 dioleate;
and
a co-solvent selected from the group consisting of PEG-400, Ethanol, and Transcutol HP.

26. The pharmaceutical composition of claim 25, wherein the capsule dosage form comprises:
active ingredients: 30-39.5% wt;
self-emulsifying system: 60-69.5% wt; and
co-solvent: 0.5-5% wt,
wherein the sum of the percentages does not exceed 100%.

27. The pharmaceutical composition of claim 25, wherein the capsule dosage form comprises:
active ingredients: 30-38% wt;
self-emulsifying system: 60-68% wt; and
co-solvent: 0.5-5% wt,
wherein the sum of the percentages does not exceed 100%.

28. The pharmaceutical composition of claim 25, wherein the co-solvent is in an amount between 0.8% and 2.5%.

29. The pharmaceutical composition of claim 25, wherein the self-emulsifying system is EG, wherein EG includes:
40-80% wt of the long-chain oil;
10-50% wt of a surfactant; and
2-25% wt of a co-surfactant,
wherein the sum of the percentages does not exceed 100%,
and wherein the surfactant is liquid at room temperature.

30. The pharmaceutical composition of claim 25, wherein the self-emulsifying system is EG, wherein EG includes:
55-70% wt of the long-chain oil;
15-40% wt of a surfactant; and
5-20% wt of a co-surfactant,
wherein the sum of the percentages does not exceed 100%,
and wherein the surfactant is liquid at room temperature.

31. The pharmaceutical composition of claim 25, wherein the self-emulsifying system is E₁, wherein E₁ includes:
5-45% wt of the long-chain oil;
45-85% wt of a surfactant; and
2-35% wt of a co-surfactant,
wherein the sum of the percentages does not exceed 100%
and wherein the surfactant is liquid at room temperature.

32. The pharmaceutical composition of claim 25, wherein the self-emulsifying system is E₁, wherein E₁ includes:
9-40% wt of the long-chain oil;
50-60% wt of a surfactant; and
10-32% wt of a co-surfactant,
wherein the sum of the percentages does not exceed 100%
and wherein the surfactant is liquid at room temperature.

33. The pharmaceutical composition of claim 25, wherein the self-emulsifying system is E₂, wherein E₂ includes:
20-60% wt of the long-chain oil;
8-40% wt of a surfactant; and
18-55% wt of a co-surfactant,
wherein the sum of the percentages does not exceed 100%
and wherein the surfactant is solid at room temperature.

34. The pharmaceutical composition of claim 25, wherein the self-emulsifying system is E₂, wherein E₂ includes:
44-55% wt of the long-chain oil;
10-15% wt of a surfactant; and
35-40% wt of a co-surfactant,
wherein the sum of the percentages does not exceed 100%
and wherein the surfactant is solid at room temperature.

35. The pharmaceutical composition of claim 25, wherein the progesterone is progesterone micronized.

36. The pharmaceutical composition of claim 25, wherein the estradiol is estradiol hemihydrate.

37. The pharmaceutical composition of claim 25, wherein the capsule dosage form includes estradiol hemihydrate, progesterone micronized, glyceryl monolinoleate, Capryolcaproyl polyoxyl-8-glycerides, propylene glycol monocaprylate, and ethanol.

38. The pharmaceutical composition of claim 25, wherein the capsule dosage form includes estradiol hemihydrate, progesterone micronized, glyceryl monolinoleate, polyoxyl-40 hydrogenated castor oil, polyglyceryl-3 dioleate and ethanol.

39. The pharmaceutical composition of claim 25, wherein the capsule dosage form is a soft gelatine capsule.

40. A method of producing a stable pharmaceutical composition comprising a capsule dosage form, comprising the steps of:
i. preparing a pre-concentrate of estradiol, wherein the preparation includes suspending all of the estradiol or a pharmaceutically acceptable salt thereof in a part of the total long-chain oil and all of the co-solvent at room temperature;
ii. separately, mixing the rest of the long-chain oil, up to 100% of the total, with the surfactant and the co-surfactant under agitation until homogenization to provide a homogenized mixture;
iii. adding the prepared pre-concentrate of step i) into the homogenized mixture of step ii) under agitation until complete solubilisation;
iv. adding progesterone or a pharmaceutically acceptable salt thereof to the solubilized estradiol solution, and mixing until homogenous suspension is obtained, and
v. optionally, encapsulating the homogeneous suspension into the capsule dosage form.

41. The process of claim 40, wherein in step ii) the surfactant is pre-heated at a temperature ≤35° C.

42. The process of claim 41, wherein the subsequent two steps iii) and iv) are performed at about the same pre-heating temperature as that of step ii), thereafter, cooling the homogeneous suspension obtained in step iv) until room temperature prior to encapsulating.

43. A stable pharmaceutical composition comprising a capsule dosage form suitable for oral administration of claim 25 which is obtained by a method comprising:
i. preparing a pre-concentrate of estradiol, wherein the preparation includes suspending all of the estradiol or a pharmaceutically acceptable salt thereof in a part of the total long-chain oil and all of the co-solvent at room temperature;
ii. separately, mixing the rest of the long-chain oil, up to 100% of the total, with the surfactant and the co-surfactant under agitation until homogenization to provide a homogenized mixture;
iii. adding the prepared pre-concentrate of step i) into the homogenized mixture of step ii) under agitation until complete solubilisation;
iv. adding progesterone or a pharmaceutically acceptable salt thereof to the solubilized estradiol solution, and mixing until homogenous suspension is obtained; and
v. optionally, encapsulating the homogeneous suspension into the capsule dosage form,
wherein the composition exhibits no presence of Impurity-M when stored for at least 3 months at 40° C./75% relative humidity (RH) condition.

44. A method of treating vasomotor symptoms (VMS) related to menopause to a patient comprising:
orally administering a stable pharmaceutical composition comprising the capsule dosage form of claim 25.
